(19) United States
(12) Patent Application Publication
(10) Pub. No.: US 2026/0193188 A1
(43) Pub. Date: Jul. 9, 2026

(54) CRYSTALLINE POLYMORPH FORMS OF A TRPV1 ANTAGONIST AND FORMULATIONS THEREOF

(71) Applicant: Bausch + Lomb Ireland Limited, Dublin (IE)

(72) Inventors: Heng GE, Jiangsu (CN); Donglei LIU, Cambridge, MA (US); Julien PAPILLON, Cambridge, MA (US); Stefan PEUKERT, Cambridge, MA (US); James J. POWERS, Cambridge, MA (US); Zhonglin SUN, Shanghai (CN)

(21) Appl. No.: 19/113,832

(22) PCT Filed: Sep. 19, 2023
(86) PCT No.: PCT/IB2023/059285

§ 371 Date: Mar. 20, 2025

(30) Foreign Application Priority Data
Sep. 21, 2022 (WO) PCT/CN2022/120270

(51) Int. Cl. C07D 217/02; A61K 9/00; A61K 31/472

(52) U.S. Cl. CPC C07D 217/02; A61K 9/0048; A61K 31/472

(57) ABSTRACT

The present disclosure provides polymorphs and formulations of 1-(6-fluoro-5-((((1r,3r)-3-(4-fluoro-3-(trifluoromethyl)phenoxy)cyclobutyl)amino)methyl)isoquinolin-8-yl)ethane-1,2-diol (compound I).

### FIELD OF THE INVENTION

The present invention relates to crystalline forms of 1-(6-fluoro-5-((((1r,3r)-3-(4-fluoro-3-(trifluoromethyl)phenoxy)cyclobutyl)amino)methyl)isoquinolin-8-yl)ethane-1,2-diol (Formula I), processes and methods for their manufacture. The invention also relates to formulations of compound I and methods for treating ocular surface disorders using same.

### BACKGROUND OF THE INVENTION

Patients suffering from ocular surface pain, particularly chronic ocular surface pain have a significant decline in quality of life, and many develop depression, moderate-to-severe angina, dialysis, disabling hip fracture and in some cases become suicidal. In many patients, the ocular surface pain remains unresolved despite treatment of the underlying pathology (e.g., recent trauma or surgery, infection, or inflammation) and other known treatments cannot be used for long term therapy.

The Transient Receptor Potential Vanilloid 1 (TRPV1) receptor is implicated in pain signaling and antagonism of this receptor may be helpful in symptoms of pain. It would be desirable to administer topically to the surface of the eye a formulation of a TRPV1 antagonist to alleviate pain, particularly chronic pain.

Formulating hydrophobic ophthalmic drugs can be particularly troublesome, because they are particularly prone to agglomeration within aqueous topical ophthalmic compositions. Agglomeration may cause stability and potentially other quality issues for the compositions, and may arise from other interactions of drugs and excipients. Accordingly, there is a need for identification of different polymorphic forms that may be formulated in ophthalmic formulations for delivery to the ocular surface.

### SUMMARY OF THE INVENTION

In one aspect, the invention relates to a crystalline form of 1-(6-fluoro-5-((((1r,3r)-3-(4-fluoro-3-(trifluoromethyl)phenoxy)cyclobutyl)amino)methyl)isoquinolin-8-yl)ethane-1,2-diol (compound I) having the structure

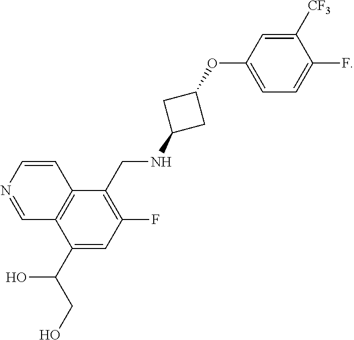 [SMILES: OCC(O)c1cc(F)c(CN[C@H]2C[C@H](Oc3ccc(F)c(C(F)(F)F)c3)C2)c2ccncc12]

In another aspect, the invention relates to a crystalline form of (S)-1-(6-fluoro-5-((((1r,3S)-3-(4-fluoro-3-(trifluoromethyl)phenoxy)cyclobutyl)amino)methyl)isoquinolin-8-yl)ethane-1,2-diol (Compound I):

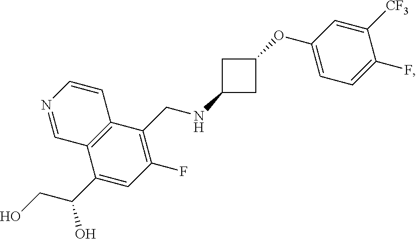 [SMILES: OC[C@@H](O)c1cc(F)c(CN[C@H]2C[C@H](Oc3ccc(F)c(C(F)(F)F)c3)C2)c2ccncc12]

described and identified herein as crystalline Form A of Compound I. Crystalline Form A of Compound I may be characterized by an X ray diffraction pattern having three or more peaks at 20 values selected from 14.3, 14.8, and 21.8±0.2° 2θ. In some embodiments, the crystalline Form A of Compound I is characterized by an X ray diffraction pattern having three or more peaks at 20 values selected from 12.5, 14.3, 14.8, 21.8, and 22.6±0.2 °2θ.

In some embodiments, crystalline Form A of Compound I is characterized by an X ray diffraction pattern as shown in FIG. 1. Crystalline Form A of Compound I may also be characterized by one of more of 1) a DSC thermogram exhibiting an endotherm at about 131.5° C.; and 2) a water loss as measured by thermogravimetric analysis of about 0.13 wt. %; and 3) a melting point of about 130.3° C.

In some embodiments, the crystalline Form A of compound I is characterized by an X ray diffraction pattern having 3 or more, 4 or more, 5 or more, 6 or more, or 7 or more peaks at 2θ values selected from 12.5, 14.3, 14.8, 20.1, 21.8, 22.6, and 23.2±0.2 °2θ.

In some embodiments, the crystalline Form A of compound I is characterized by an X ray diffraction pattern having 3 or more, 4 or more, 5 or more, 6 or more, or 7 or more peaks at 2θ values selected from 7.1, 12.5, 14.3, 14.8, 18.7, 20.1, 21.8, 22.6, 23.2, 25.1, and 27.9 0.2 °2θ.

In another aspect, the present invention provides a method of preparing a crystalline Form A of Compound I, comprising cooling a hot saturated solution of the free base of Compound I in a solvent, to crystallize Compound I as crystalline Form A.

In another aspect, the present invention provides a method of preparing a crystalline Form A of Compound I, comprising crystallizing form A from a solution of compound I in a solvent, e.g., at room temperature.

In another aspect, the present invention provides a method of preparing a crystalline Form A of Compound, comprising adding an antisolvent to a solution of compound I in a solvent.

In a particular embodiment of any of the methods of preparation of crystalline Form A of Compound I, compound I is used as the free base.

In some embodiments, crystalline Form A of Compound I is characterized by a melting point of about 130.3° C. or a differential scanning calorimetry pattern as shown in FIG. 2.

In another aspect, the invention provides a pharmaceutical formulation, comprising the crystalline Form A in substantially pure form.

In a further aspect, a method of preparing a pharmaceutical formulation comprising crystalline Form A is provided, the method comprising dissolving crystalline Form A as disclosed herein in an ophthalmically acceptable carrier formulated for ocular use (e.g., topical application to the ocular surface).

In yet another aspect, the present invention provides a method of treating a TRPV1-mediated disease or disorder in a subject in need thereof, the method comprising administering to the subject a pharmaceutical formulation comprising an effective amount of Compound I, or a pharmaceutically acceptable salt, solvate, or co-crystal thereof, prepared from the crystalline Form A of Compound I, or a crystalline Form A of Compound I, or a combination thereof.

In yet another aspect, the present invention provides a method of treating an ocular surface disorder in a subject in need thereof, the method comprising administering to the subject a pharmaceutical formulation comprising an effective amount of Compound I, or a pharmaceutically acceptable salt, solvate, or co-crystal thereof, prepared from the crystalline Form A of Compound I, or a crystalline Form A of Compound I, or a combination thereof.

In yet another aspect, the present invention provides a method of treating ocular surface pain in a subject in need thereof, the method comprising administering to the subject a pharmaceutical formulation comprising an effective amount of Compound I, or a pharmaceutically acceptable salt, solvate, or co-crystal thereof, prepared from the crystalline Form A of Compound I, or a crystalline Form A of Compound I, or a combination thereof.

In some embodiments, the present disclosure is related to a method of treating ocular surface pain in a subject in need thereof, comprising ocularly administering an effective amount of 1-(6-fluoro-5-((((1r,3r)-3-(4-fluoro-3-(trifluoromethyl)phenoxy)cyclobutyl)amino)methyl)isoquinolin-8-yl)ethane-1,2-diol (compound of formula I) having structure:

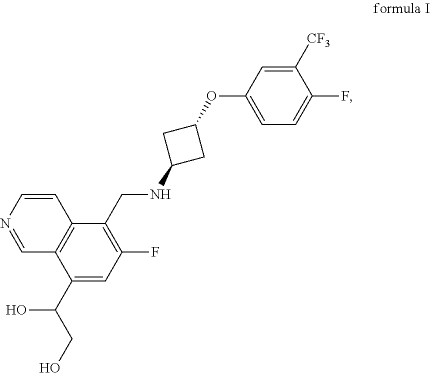 [SMILES: OCC(O)c1cc(F)c(CN[C@H]2C[C@H](Oc3ccc(F)c(C(F)(F)F)c3)C2)c2ccncc12]

or a pharmaceutically acceptable salt, solvate, polymorph, or co-crystal thereof to the subject.

In some embodiments, the compound of formula I has the structure:

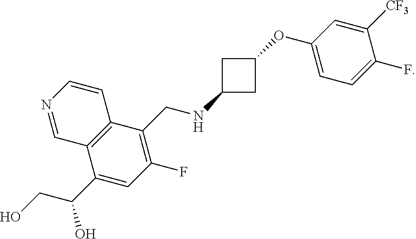 [SMILES: OC[C@@H](O)c1cc(F)c(CN[C@H]2C[C@H](Oc3ccc(F)c(C(F)(F)F)c3)C2)c2ccncc12]

In some embodiments, the ocular surface pain is acute or episodic ocular surface pain.

In some embodiments, the ocular surface pain is chronic ocular surface pain lasting for at least 3 months. In some embodiments, the compound of Formula I is administered to the cornea of the subject.

In some embodiments, the COSP is associated with dry eye disease. In some embodiments, the administration results in a decrease in the symptoms of dry eye disease. In some embodiments, the administration results in a decrease in the pain associated with dry eye disease. In some embodiments, the administration results in reduced incidence of at least about 10% in one or more of ocular dryness, ocular discomfort, ocular hyperemia, ocular burning or stinging, grittiness or foreign body sensation, or photophobia.

In some embodiments, the subject suffers from one or more of dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, or patients recovering from neurotrophic keratitis.

In some embodiments, the method comprises administering an additional therapeutic agent to the subject.

In some embodiments, the administration results in a reduction in a pain score on the visual acuity scale (VAS) of at least about 3, at least about 4, at least about 5, at least about 6, at least about 7, at least about 8, at least about 9 or at least about 10, compared to a placebo. In some embodiments, the reduction in VAS score arises from the difference in VAS scores prior to and after administration of compound I to the subject. The method according to the invention, wherein the reduction in VAS score occurs within about half hour, about one hour, within about 2 hours, within about 4 hours, or about 2-4 hours after administration of compound I to the subject.

In some embodiments, the administration of compound I results in a reduction in hyperemia in the subject of at least about 1, at least about 2, at least about 3, at least about 4, or at least about 5, on the McMonnies scale.

In some embodiments, the administration does not result in a change in one or more of best corrected visual acuity, intraocular pressure, slit-lamp biomicroscopy, dilated eye exam, blink rate, tear production, corneal staining, compared to a placebo.

In some embodiments, the compound of formula I is administered in the form of a formulation as described herein. In some embodiments, the formulation is administered for at least about one, about two, or about three months. In some embodiments, the formulation is administered one to four times daily.

In some embodiments, the disclosure provides a formulation as described herein, for use in the treatment of ocular surface pain. In some embodiments of the described uses, the ocular surface pain is episodic (e.g., acute) ocular surface pain or chronic ocular surface pain lasting for at least 3 months.

In some embodiments, the disclosure provides a method of reducing ocular surface pain in a subject in need thereof, comprising ocularly administering 1-(6-fluoro-5-((((1r,3r)-3-(4-fluoro-3-(trifluoromethyl)phenoxy)cyclobutyl)amino)methyl)isoquinolin-8-yl)ethane-1,2-diol (Formula I) having structure:

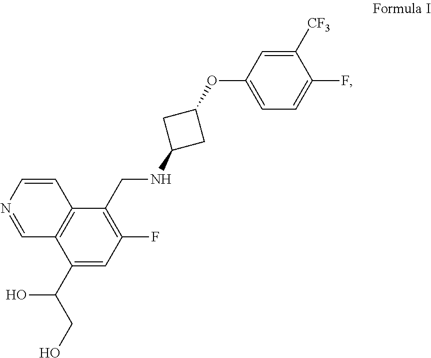 [SMILES: OCC(O)c1cc(F)c(CN[C@H]2C[C@H](Oc3ccc(F)c(C(F)(F)F)c3)C2)c2ccncc12]

or a pharmaceutically acceptable salt, solvate, polymorph, or co-crystal thereof to the subject.

In some embodiments, the compound of formula I has the structure:

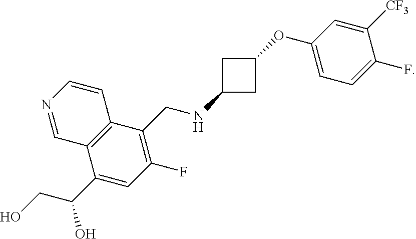 [SMILES: OC[C@@H](O)c1cc(F)c(CN[C@H]2C[C@H](Oc3ccc(F)c(C(F)(F)F)c3)C2)c2ccncc12]

In some embodiments, the ocular surface pain is episodic (e.g., acute) ocular surface pain the ocular surface pain is chronic ocular surface pain (COSP). In some embodiments, the COSP is associated with dry eye disease.

In some embodiments, the administration results in a decrease in the symptoms of dry eye disease. In some embodiments, the administration results in a decrease in the pain associated with dry eye disease. In some embodiments, the administration results in reduced incidence of at least about 10% in one or more of ocular dryness, ocular discomfort, ocular hyperemia, ocular burning or stinging, grittiness or foreign body sensation, or photophobia.

In some embodiments, the subject suffers from one or more of dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, or patients recovering from neurotrophic keratitis.

In some embodiments, the method comprises administering an additional therapeutic agent to the subject.

In some embodiments, the administration results in a reduction in a pain score on the visual acuity scale (VAS) of at least about 3, at least about 4, at least about 5, at least about 6, at least about 7, at least about 8, at least about 9 or at least about 10, compared to a placebo. In some embodiments, the administration results in a reduction in a VAS pain score of at least about 6, at least about 7, at least about 8, at least about 9 or at least about 10, compared to a placebo. In some embodiments, the reduction in the pain score arises from the difference in pain scores prior to and after administration of compound I to the subject.

In some embodiments, the administration results in a reduction in hyperemia in the subject of least about 1, at least about 2, at least about 3, at least about 4, or at least about 5, on the McMonnies scale.

In some embodiments, the administration results in a reduction in a pain score on the visual acuity scale (VAS) of at least about 3 as compared to a VAS score prior to administration of the compound.

In some embodiments of the recited methods, the compound of formula I is administered in the form of a formulation as described herein.

Specific preferred embodiments of the invention will become evident from the following more detailed description of certain preferred embodiments and the claims.

### BRIEF DESCRIPTION OF THE DRAWINGS

FIG. 1 provides the X-ray powder diffraction pattern of crystalline Form A of compound I.

FIG. 2 provides a differential scanning calorimetry scan of crystalline Form A of compound I.

FIG. 3 provides a thermogravimetric analysis of crystalline Form A of compound I.

FIG. 4 provides XRPD patterns of compound I form A after grinding and granulation, from bottom to top: starting material, after grinding, after granulation with water, after granulation with ethanol.

### DETAILED DESCRIPTION

For manufacturing pharmaceutical compounds and their formulations, it is important that the active compound is in a form that can be conveniently handled and processed in order to obtain a commercially viable, reliable, and reproducible manufacturing process.

It has been surprisingly found that crystalline Form A of Compound I possesses favorable physicochemical properties which are particularly useful for a drug substance intended for use in ophthalmic dosage form preparation.

As used herein, the term “about” refers to a range of values +/−10% of a specified value.

“TRPV1 receptor” refers to the Transient Receptor Potential Vanilloid 1 that has been characterized through molecular cloning and pharmacology. See e.g., Caterina M J, et al., Nature 1997; 389:816-824. TRPV1 receptor activity is measured as described in WO2005/120510, hereby incorporated by reference in its entirety.

The language “effective amount” of the compounds described herein, refers to that amount of a therapeutic compound necessary or sufficient to perform its intended function within a mammal. An effective amount of the therapeutic compound can vary according to factors such as the amount of the causative agent already present in the mammal, the age, sex, and weight of the mammal, and the ability of the therapeutic compounds of the present disclosure to treat the ocular surface disorder and/or symptoms thereof in the mammal.

The phrase “ophthalmically compatible” refers to formulations, polymers and other materials and/or dosage forms which are suitable for use in contact with the ocular tissues of human beings and animals without excessive toxicity, irritation, allergic response, or other problem or complication, commensurate with a reasonable benefit/risk ratio.

As used herein, the term “treat”, “treating” or “treatment” in connection to a disease or disorder refers in some embodiments, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder or symptom thereof.

As used herein, the term “subject” or “patient” refers to human and non-human mammals, including but, not limited to, primates, rabbits, pigs, horses, dogs, cats, sheep, and cows. In particular embodiments, a subject or patient is a human. In some embodiments, the term “patient” or “subject” refers to a human being who is diseased with the condition (i.e., disease or disorder) described herein and who would benefit from the treatment. As used herein, a subject is “in need of” a treatment if such subject (patient) would benefit biologically, medically or in quality of life from such treatment. In particular embodiments, the subject is an adult human of at least about 18 years of age. In some embodiments, the subject is an adult human from about 18 years of age to about 75 years of age. In some embodiments, the subject is a child of up to about 18 years of age.

As used herein, “ocular surface” refers to the outer surface of the eye, which anatomically comprises the cornea (with epithelium, bowman layer, stroma, descement membrane, endothelium), conjunctiva, and the corneo-scleral junction, i.e. limbus.

As used herein, “pain” refers to constant or intermittent sensation of actual pain described as but not limited to stabbing, dull, sharp, or ache. Pain may also refer to similar related descriptors such as but not limited to burning, stinging, grittiness, foreign body sensation, dryness, sandy, tired, itchy, irritated, sensitivity to light.

As used herein, “ocular surface pain” refers to pain on the surface of the eye, e.g., cornea. Ocular pain may be nociceptic pain, which is generally caused by external physical or chemical damaging stimuli such as corneal surgery, inflammation, or other damage to the corneal surface. Ocular pain may also result from neuropathic pain, which may occur due to direct damage to the neurons of the body, resulting in messages of pain being sent to the central nervous system and brain regardless of the presence of noxious stimuli. As used herein “ocular surface pain” includes both nociceptic pain and neuropathic pain.

As used herein, the term “visual analog scale” (VAS) is a measure of pain intensity where a subject typically marks a place on a scale that aligns with their level of pain. The pain is marked in a range of “no pain” (score of 0) and “pain as bad as it could be” or “worst imaginable pain” (score of 100). See e.g., Hawker, et al., Arthritis Care & Research 63(11), pp. S240-S252 (November 2011). There are several other well-designed pain scales that may be used to help assess the extent of pain. The numerical rating scale (NRS) is often used, in which subjects use numbers to rate pain. The number scale may be from 1-10, or 1-100. The Wong-Baker FACES Pain Scale combines pictures and numbers for pain ratings. It can be used in children over the age of 3 and in adults. Six faces depict different expressions, ranging from happy to extremely upset. Each is assigned a numerical rating between 0 (smiling) and 10 (crying). The Verbal Pain Intensity Scale uses wordings on a scale to rate pain intensity: No Pain/Mild Pain/Moderate Pain/Severe Pain Very Severe Pain/Worst Possible Pain.

The Eye Sensation Scale is a specific pain scale was developed to measure ophthalmic pain severity. See Caudle L. E. et al., Optom Vis Sci. 2007 August; 84(8):752-62. In this scale, pain, discomfort or light sensitivity is typically measured by 5 category labels of “extreme,” “severe,” “moderate,” “mild,” or “none.”

The Ocular Pain Assessment Survey (OPAS) is a quantitative, multidimensional questionnaire, specifically designed for assessment of corneal and ocular surface pain and Quality of Life (QoL) changes. The OPAS assesses pain intensity, frequency of eye and non-eye pain, QoL changes, aggravating factors, associated factors, and symptomatic relief quantitative, allowing for monitoring of treatment responses. See Qazi et al., Ophthalmology July 123(7):1458-1468 (2016).

As used herein, ocular hyperemia refers to redness of the ocular surface. Ocular hyperemia may be a clinical marker for inflammation and/or ocular irritation. Ocular hyperemia is typically measured using the McMonnies scale, at values from 0 to 5, based on standard photographs.

As used herein, “placebo” refers to an ophthalmic formulation that includes all the components of the administered drug composition without the drug.

As used herein “polymorph” refers to crystalline forms having the same chemical composition but different spatial arrangements of the molecules, atoms, and/or ions forming the crystal.

As used herein “solvate” refers to a crystalline form of a molecule, atom, and/or ions that further comprises molecules of a solvent or solvents incorporated into the crystalline lattice structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate. Solvates may occur as dimers or oligomers comprising more than one molecule of compound I within the crystalline lattice structure.

As used herein “amorphous” refers to a solid form of a molecule, atom, and/or ions that is not crystalline. An amorphous solid does not display a definitive X-ray diffraction pattern.

As used herein, “substantially pure,” when used in reference to a form, means a compound having a purity greater than 90 weight %, including greater than 90, 91, 92, 93, 94, 95, 96, 97, 98, and 99 weight %, and also including equal to about 100 weight % of compound I, based on the weight of the compound. The remaining material comprises other form(s) of the compound, and/or reaction impurities and/or processing impurities arising from its preparation. For example, a crystalline form of compound I may be deemed substantially pure in that it has a purity greater than 90 weight %, as measured by means that are at this time known and generally accepted in the art, where the remaining less than 10 weight % of material comprises other form(s) of compound I and/or reaction impurities and/or processing impurities. In some embodiments, the crystalline form A of Compound I has a purity greater than 92 weight %. In some embodiments, the crystalline form A of Compound I has a purity greater than 95 weight %. In some embodiments, the crystalline form A of Compound I has a purity greater than 97 weight %. In some embodiments, the crystalline form A of Compound I has a purity greater than 99 weight %.

The term “substantially free of chloride” means that the compound, e.g., crystalline Form A of Compound I, contains no significant amount of extraneous chloride, e.g., undesired chloride ions as a result of hydrochloride salt formation during drug substance manufacture. In some embodiments, crystalline Form A contains less than 0.5 weight % chloride ions. In some embodiments, crystalline Form A contains less than 0.3 weight % chloride ions. In some embodiments, crystalline Form A contains less than 0.1 weight % chloride ions. In a particular embodiment, crystalline Form A contains less than 0.05 weight % chloride ions.

As used herein, “Compound of formula I,” “Compound I,” “Formula I,” and “compound I” are used interchangeably and mean a compound that has the name 1-(6-fluoro-5-((((1r,3r)-3-(4-fluoro-3-(trifluoromethyl)phenoxy)cyclobutyl)amino)methyl)isoquinolin-8-yl)ethane-1,2-diol, the structure shown below:

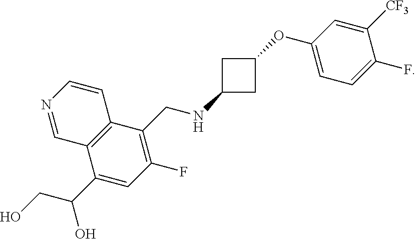 [SMILES: OCC(O)c1cc(F)c(CN[C@H]2C[C@H](Oc3ccc(F)c(C(F)(F)F)c3)C2)c2ccncc12]

In a particular embodiment, Compound I has the name (S)-1-(6-fluoro-5-((((1r,3S)-3-(4-fluoro-3-(trifluoromethyl)phenoxy)cyclobutyl)amino)methyl)isoquinolin-8-yl)ethane-1,2-diol, and has the structure shown below:

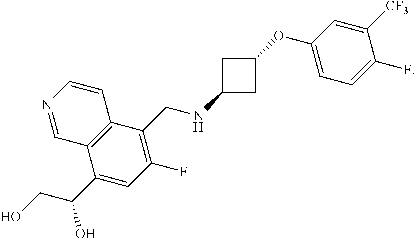 [SMILES: OC[C@@H](O)c1cc(F)c(CN[C@H]2C[C@H](Oc3ccc(F)c(C(F)(F)F)c3)C2)c2ccncc12]

As used herein, “crystal form,” “crystalline form,” “modification,” or “polymorph,” or “polymorphic form” in upper or lower case are used interchangeably and refer to the crystalline or polymorphic form of compound I.

As described here, compound of formula I is a stereoisomer that may be present either in its racemic form or in an enantiomeric excess (ee) of the isomer shown above, wherein the isomer shown above is present in at least 90% ee, at least 95% ee, at least 96% ee, at least 97% ee, at least 98% ee, or at least 99% ee.

Any chemical formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the disclosure include, for example, isotopes of hydrogen, carbon, nitrogen, and oxygen, such as ²H, ³H, ¹¹C, ¹³C, ¹⁴C, and ¹⁵N. Accordingly, it should be understood that methods of the present invention can or may involve compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically-labeled compounds can generally be prepared by conventional techniques known to those skilled in the art, e.g., using an appropriate isotopically-labeled reagents in place of the non-labeled reagent previously employed.

The present invention encompasses embodiments that include all pharmaceutically acceptable salts of the compounds useful according to the invention provided herein. As used herein, “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts of the parent compound formed, for example, from non-toxic inorganic or organic acids. The pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety. For example, preferred pharmaceutically acceptable salts include, but are not limited to, mineral or organic acid salts of basic residues such as amines. For example, the salt can be a hydrochloride salt.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Unless indicated otherwise, all ingredient concentrations are presented in units of % weight/volume (% w/v).

Unless otherwise specified, the weight or dosage referred to herein for the compound of formula I is the weight or dosage of the compound itself, not that of a salt or prodrug thereof, which can be different to achieve the intended therapeutic effect. For example, the weight or dosage of a corresponding salt of a compound suitable for the methods, compositions, or combinations disclosed herein may be calculated based on the ratio of the molecular weights of the salt and compound itself.

### Crystalline Forms of Compound I

Polymorphism is the ability of solid materials to exist in two or more crystalline forms with different arrangements or conformations of the constituents in the crystal lattice. Polymorphism and pseudomorphism are very common amongst drugs and are responsible for differences in many properties. While convention dictates selection of the lowest energy polymorph for incorporation into a formulation due to its chemical stability, considerations must be given to the excipients in the formulation to achieve desired chemical and physical stability and therefore efficacy. Crystalline Form A of Compound I can be advantageously used to prepare or incorporated into ophthalmic formulations for the treatment of TRPV1-mediated disorders. Crystalline Form A of Compound I can be advantageously used to prepare or incorporated into ophthalmic formulations for the treatment of ocular surface pain. In some embodiments, the subject suffers from one or more of dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, or patients recovering from neurotrophic keratitis.

Therefore, in one aspect, the present invention provides crystalline Form A of Compound I. Crystalline Form A of Compound I is a non-solvated crystalline solid and crystallizes in two distinctly different morphologies. Non-aqueous solvent systems yield irregularly shaped tabular particles, while solvent mixtures containing water result in acicular particles. Despite the change in morphology, both forms have the same physicochemical characteristics and the same XRD pattern and are therefore considered to be the same polymorph. Crystalline Form A may be characterized as such by powder X-ray diffraction (XRPD) wherein the pattern resulting from the analysis comprises significant peaks at characteristic 2-theta (2θ) angles. Form A may be characterized, for example, by an X ray diffraction pattern having three or more peaks at 2θ values selected from 14.3, 14.8, and 21.8±0.2 °2θ. Form A may be characterized, for example, by an X ray diffraction pattern having three or more peaks at 2θ values selected from 12.5, 14.3, 14.8, 21.8, and 22.6±0.2 °2θ. Yet further, Form A may be characterized, for example, by an X-ray diffraction pattern having 3 or more, 4 or more, 5 or more, 6 or more, or 7 or more peaks at 2θ values selected from 12.5, 14.3, 14.8, 20.1, 21.8, 22.6, and 23.2±0.2 °2θ. As described herein, the X-ray diffraction peaks are as measured using CuK_{α} radiation having a wavelength of 0.15418 nm. Parameters that may be used to analyze Compound I by XRPD may be found in the General test conditions in the Examples section disclosed herein. In some embodiments, crystalline Form A is characterized by a melting point of about 130.3° C.

Crystalline Form A may be prepared by cooling a hot saturated solution of compound I in a solvent, to crystallize compound I as crystal form A. In another embodiment, crystalline Form A may be prepared by crystallizing form A from a saturated solution of compound I in a solvent.

In some embodiments, crystalline Form A of Compound I may be prepared by dissolving an appropriate amount Compound I in a minimal amount of solvent, to ensure saturation, at a temperature of about 50° C. to 70° C., e.g., 60° C. The solution may then be slowly cooled to a temperature of between about 0° C. to about 30° C., e.g., 20° C., during which period the solution may be stirred. The solution may optionally be cooled at a temperature of between about −20° C. to about −5° C. over a period of about 5 days, in order to aid the crystallization process. Crystalline Form A of Compound I may then be isolated, for example, by filtering the suspension to isolate a solid which has formed. The solid may be dried, for example, under vacuum at about 45 to about 55° C. for about 4 hours to about 5 hours to yield Compound I in its polymorph Form A crystalline form.

In some embodiments, the solvent is selected form the group consisting of acetone, acetonitrile, dichloromethane, ethanol, ethyl acetate, isopropyl acetate, 2-methyl-2-butanol, methyl tert-butyl ether, water, methanol/water and acetone/heptane.

In some embodiments, crystalline Form A of Compound I may be prepared by suspending Compound I in a solvent, and stirring the mixture. The mixture may be stirred over a period of time, for example, up to 28 days. Any solids can be optionally filtered and the mother liquor may be left at about 25° C. to evaporate. For non-volatile solvents (e.g., benzyl alcohol), the solution may be stored at −20° C. for at least 1 day (24 hours). Crystalline Form A of Compound I may then be isolated, for example, by filtering the reaction mixture to isolate a solid which has formed. The solid may be dried, for example, under vacuum at about 45 to about 55° C. for about 4 hours to about 5 hours to yield Compound I in its polymorph Form A crystalline form.

In some embodiments, the solvent is selected from the group consisting of acetone, acetonitrile, benzyl alcohol, dichloromethane, dioxane, ethanol, ethyl acetate, isopropyl acetate, methanol, 2-methyl-2-butanol, methyl tert-butyl ether, tetrahydrofuran, 1-propanol/water, 2-propanol/water and methanol/water.

Crystalline Form A of Compound I may also be prepared by suspending Compound I in a solvent, and heating to a temperature such that it does not exceed the boiling point of the solvent. The reaction may be heated and stirred to obtain a solution. For example, if the solvent is acetonitrile, the suspension is heated to a temperature of between about 50 and 70° C., e.g., 55° C. Heating may be carried out until dissolution is achieved, for example, 2 hours to 3 hours. The starting Compound I material may be any form (e.g., crystalline, amorphous, solvated) to form a reaction mixture.

The solution may then be cooled to a lower temperature to yield Compound I in its polymorph Form A crystalline form.

The cooling may be carried out in a step-wise fashion over a period of time up to 1 day (24 hours). For example, the solution at 55° C. may be slowly cooled to a temperature of about 50° C. The cooling may take place over a period of time, for example, of about 1 hour. The reaction mixture may be maintained at this temperature for a prolonged period, for example, about 3 hours to about 4 hours.

In a second step, the reaction mixture may be further slowly cooled to about 25° C. to about 30° C., for example, over a period of about 5 hours to about 6 hours. The reaction mixture may subsequently be heated to 50° C., and maintained at this temperature for a prolonged period, for example, over a period of about 3 hours to about 4 hours, during which the reaction mixture may be stirred.

In a third cooling step, the reaction mixture may then be further slowly cooled to about 10° C. to about 15° C. The reaction mixture may be maintained at this temperature for a prolonged period, for example, for about 5 hours to about 6 hours, during which the reaction mixture may be stirred.

Crystalline Form A of Compound I may then be isolated, for example, by filtering the reaction mixture to isolate a solid which has formed. The solid may be dried, for example, under vacuum at about 45 to about 55° C. for about 4 hours to about 5 hours to yield Compound I in its polymorph Form A crystalline form.

Optionally, a further purification step may be performed to removal residual chloride ions. Crystalline Form A of Compound I is suspended in water and stirred at 25° C. for a prolonged period, for example for about 5 hours to about 7 hours, e.g., about 6 hours. In a next step, the reaction mixture is filtered and the solid is re-suspended in water. The process may be repeated at least four times. The residual chloride content may be determined by ion chromatography. The crystalline Form A of Compound I may then be isolated, for example, by filtering the reaction mixture to isolate the solid. The solid may be dried, for example, under vacuum at about 45 to about 55° C., e.g., at about 50° C., for about 5 hours to about 6 hours to yield Compound I in its Form A crystalline form, which is substantially free of chloride.

Alternatively, crystalline Form A may be prepared by adding an antisolvent to a solution of compound I in a solvent. In a particular embodiment, the antisolvent is selected from heptane, toluene, and water. In a further embodiment the antisolvent/solvent system is selected from the group consisting of heptane/acetone, water/acetone, heptane/dioxane, toluene/dioxane, water/dioxane, heptane/ethanol, water/ethanol, heptane/ethyl acetate, water/methanol, heptane/2-methyl-2-butanol, heptane/tetrahydrofuran, and toluene/tetrahydrofuran.

Crystalline Form A of Compound I may be prepared by dissolving an appropriate amount Compound I in a minimal amount of solvent, to ensure saturation, and adding said solution into an excess of antisolvent, during which period the reaction mixture may be stirred. In the case there is no immediate precipitation, the reaction mixture may be kept under stirring at room temperature for 1 day (24 hours). Crystalline Form A of Compound I may then be isolated, for example, by filtering the reaction mixture to isolate a solid which has formed. The solid may be dried, for example, under vacuum at about 45 to about 55° C. for about 4 hours to about 5 hours to yield Compound I in its polymorph Form A crystalline form.

In some embodiments, Compound I useful in the preparation of the crystalline Form A is substantially pure.

In one embodiment, a crystalline Form A of compound I is provided in substantially pure form. This crystalline Form A of compound I in substantially pure form may be employed in pharmaceutical compositions, e.g., ophthalmic formulations as described herein. In some embodiments, the disclosure provides for pharmaceutical formulations comprising Compound I in crystalline Form A. In some embodiments, the disclosure provides for pharmaceutical formulations comprising Compound I, or a pharmaceutically acceptable salt, solvate, or co-crystal thereof, prepared from the crystalline Form A of Compound I.

### Formulations

Some embodiments herein are directed to a pharmaceutical formulation comprising the crystalline Form A of Compound I or a pharmaceutical formulation prepared from the crystalline polymorph Form A of Compound I.

In some embodiments, the formulation further includes at least one ophthalmically acceptable excipient.

In some embodiments, the invention provides for the use of crystalline Form A of Compound I, in the preparation of a pharmaceutical formulation.

In some embodiments, the invention provides a method of preparing a pharmaceutical formulation comprising crystalline Form A of Compound I, the method comprising dissolving the crystalline Form A of Compound I in an ophthalmically acceptable carrier formulated for ocular use, e.g., for topical application to the ocular surface.

In some embodiments, the formulation includes a buffer. Examples of buffer substances include acetate, ascorbate, borate, hydrogen carbonate, carbonate, citrate, edetate (EDTA) gluconate, lactate, phosphate, propionate and TRIS (tromethamine) buffers. In particular embodiments, the buffer is a phosphate buffering system. In particular embodiments, the buffer is a tromethamine buffer. The amount of buffer substance added is, typically, that necessary to ensure and maintain a physiologically tolerable pH range. In some embodiments, the pH range is in the range of from about 4 to about 9, from about 4.5 to about 8.5, from about 5.0 to about 8.0, from about 5.5 to about 8.0, from about 6.4 to about 8.4. In some embodiments, the pH is about 6.0. In particular embodiments, the pH is about 7.4.

In some embodiments, the formulation may also be self-preserved and does not include a preservative. In other embodiments, the formulation includes a preservative. In some embodiments, the preservative includes, without limitation, polyhexylmethylene biguanidine (PHMB), polymeric quaternary ammonium compound (e.g., polyquatemium-1), chlorine containing preservatives such as benzalkonium chloride (BAK), chlorite preservatives or others.

In some embodiments, the preservative is polymeric quaternary ammonium compounds that are ophthalmically acceptable. Compounds of this type are described in U.S. Pat. Nos. 3,931,319; 4,027,020; 4,407,791; 4,525,346; 4,836,986; 5,037,647 and 5,300,287; and PCT application WO 91/09523 (Dziabo et al.). In particular embodiments, the polymeric ammonium compound is polyquaternium 1, otherwise known as POLYQUAD® or ONAMERM® with a number average molecular weight between 2,000 to 30,000. In still particular embodiments, the number average molecular weight is between 3,000 to 14,000.

When used, the polymeric quaternary ammonium compound is generally used in an amount that is greater than about 0.00001 w/v %, greater than about 0.0003 w/v %, or greater than about 0.0007 w/v % of the formulation. Moreover, the polymeric quaternary ammonium compound, when used in the formulation, is generally used at a concentration that is less than about 0.03 w/v %, less than about 0.003 w/v %, or less than about 0.0015 w/v % of the formulation. In some embodiments, the concentration of polymeric quaternary ammonium compound in the formulation are as follows: greater than about 0.0003 w/v % but less than about 0.003 w/v %; greater than about 0.0003 w/v % but less than about 0.0015 w/v %; greater than about 0.0007 w/v % but less than about 0.003 w/v %; and greater than about 0.0007 w/v % but less than about 0.0015 w/v %. In particular embodiments, the formulation includes polyquarternium 1 at a concentration of about 0.0010% w/v.

In some embodiments, the formulation includes BAK at a concentration that is at least about 0.0005 w/v %, about 0.001 w/v %, or greater than about 0.007 w/v % of the formulation, and at a concentration that is less than about 0.1 w/v %. less than about 0.02 w/v %, or less than about 0.0035 w/v % of the ophthalmic composition. It is specifically contemplated that any of the lower limits on the concentration of BAK may be used in conjunction with any of the upper limits on the concentrations of BAK. In particular embodiments, the concentration of BAK in the composition are as follows: greater than about 0.001 w/v % but less than about 0.02 w/v %; greater than about 0.001 w/v % but less than about 0.0035 w/v %; greater than about 0.007 w/v % but less than about 0.02 w/v %; and greater than about 0.007 w/v % but less than about 0.0035 w/v %.

In some embodiments, the formulations of the invention may include an additional therapeutic agent in addition to compound I. Further therapeutic agents may include, for instance, other compounds and antibodies useful for treating ocular surface disorders. A non-limiting list of such agents incudes nonsteroidal anti-inflammatory drugs such as ketorolac, nepafenac, bromfenac, corticosteroids; drugs for dry eye disease such as cyclosprine, lifitegrast, or other TRPV1 inhibitors.

In some embodiments, the formulation is stored at refrigerated temperatures (e.g., 4° C.). In some embodiments, the formulation is warmed to room temperature prior to administration.

In some embodiments, the suspension is packaged in a single dose container. In some embodiments, the formulation is packaged in a multi-dose container.

The formulations described herein are delivered to the surface of the eye one to six times a day, depending on the routine discretion of the skilled clinician. In some embodiments, the formulations are administered, one, two, three, or four times a day.

In some embodiments, the pharmaceutical formulations of the invention may include an additional therapeutic agent in addition to Compound (I). Further therapeutic agents may include, for instance, other compounds and antibodies useful for treating ocular surface disorders. A non-limiting list of such agents incudes nonsteroidal anti-inflammatory drugs such as ketorolac, nepafenac, bromfenac, corticosteroids; drugs for dry eye disease such as cyclosporine, lifitegrast, or other TRPV1 inhibitors.

### Methods of Use

Without being bound by theory, it is hypothesized that blockers of the Transient Receptor Potential Vanilloid 1 (TRPV1) receptor may be useful in the treatment of pain, e.g., chronic pain.

Accordingly, in some embodiments, the invention provides a method of treating ocular surface pain in a subject, said method includes administering to the subject an effective amount of compound (I), or a pharmaceutically acceptable salt, solvate, or co-crystal thereof. In some embodiments, the method comprises administering compound (I), or a pharmaceutically acceptable salt, solvate, or co-crystal thereof as a pharmaceutical formulation, for example, as disclosed herein. In some embodiments, the pharmaceutical formulation is prepared from the crystalline polymorph Form A of Compound I as disclosed herein. In some embodiments, the invention provides a method of reducing ocular surface pain in a subject in need thereof, said method includes administering to the subject an effective amount of compound (I), or a pharmaceutically acceptable salt, solvate, or co-crystal thereof. In some embodiments, the method comprises administering compound (I), or a pharmaceutically acceptable salt, solvate, or co-crystal thereof as a pharmaceutical formulation, for example, as disclosed herein. In some embodiments, the pharmaceutical formulation is prepared from the crystalline polymorph Form A of Compound I as disclosed herein. In some embodiments, the invention provides a method of treating an ocular surface disorder in a subject, said method includes administering to the subject an effective amount of compound (I), or a pharmaceutically acceptable salt, solvate, or co-crystal thereof. In some embodiments, the method comprises administering compound (I), or a pharmaceutically acceptable salt, solvate, or co-crystal thereof as a pharmaceutical formulation, for example, as disclosed herein. In some embodiments, the pharmaceutical formulation is prepared from the crystalline polymorph Form A of Compound I as disclosed herein.

In some embodiments, the invention provides for the use of the compound of formula I, or a pharmaceutically acceptable salt, solvate, or co-crystal thereof, in the treatment or reduction of ocular surface pain.

In some embodiments, the compound of formula I is in polymorphic Form A.

In particular embodiments, the methods described herein are carried out by administering the formulations of compound I described supra. Thus, the invention provides a method of treating ocular surface pain by administering a formulation of compound I as described herein. In some embodiments, the method results in a reduction in ocular surface pain.

In some embodiments, the invention provides a pharmaceutical formulation comprising compound (I) or a pharmaceutically acceptable salt, solvate, or co-crystal thereof, for use in the treatment of ocular surface pain. In an embodiment, the formulation is prepared from the crystalline polymorph Form A of Compound I as disclosed herein.

In some embodiments, the invention provides a pharmaceutical formulation comprising crystalline Form A of Compound I, for use in the treatment of ocular surface pain.

In some embodiments, the invention provides a pharmaceutical formulation comprising compound (I) or a pharmaceutically acceptable salt, solvate, or co-crystal thereof, for use in the treatment of an ocular surface disorder. In an embodiment, the formulation is prepared from the crystalline polymorph Form A of Compound I as disclosed herein.

In some embodiments, the invention provides a pharmaceutical formulation comprising crystalline Form A of Compound I, for use in the treatment of an ocular surface disorder.

In some embodiments, the invention provides a pharmaceutical formulation comprising compound (I) or a pharmaceutically acceptable salt, solvate, or co-crystal thereof, for use in reducing ocular surface pain. In an embodiment, the formulation is prepared from the crystalline polymorph Form A of Compound I as disclosed herein.

In some embodiments, the invention provides a pharmaceutical formulation comprising crystalline Form A of Compound I, for use in reducing ocular surface pain.

In some embodiments, the invention provides for the use of crystalline Form A of Compound I as disclosed herein, in the manufacture of a medicament for the treatment of ocular surface pain.

In some embodiments, the subject suffers from episodic or acute ocular pain. In some embodiments, the subject suffers from chronic ocular surface pain, which lasts for at least three months. In some embodiments, the subject suffers from chronic ocular surface pain, which lasts for at least two months. In some embodiments, the subject suffers from chronic ocular surface pain, which lasts for at least one month. In some embodiments, the subject suffers from chronic ocular surface pain, which lasts for at least four months. In some embodiments, the subject suffers from chronic ocular surface pain, which lasts for at least five months. Thus, in some embodiments, the invention provides a method of treating chronic ocular surface pain in a subject by administering to the subject an effective amount of compound of formula I, or a pharmaceutically acceptable salt, solvate, polymorph, or co-crystal thereof. In some embodiments, the invention provides a method of reducing chronic ocular surface pain in a subject by administering to the subject an effective amount of compound of formula I, or a pharmaceutically acceptable salt, solvate, polymorph, or co-crystal thereof. The invention provides for the use of the compound of formula I, or a pharmaceutically acceptable salt, solvate, polymorph, or co-crystal thereof, in the treatment of chronic ocular surface pain. In some embodiments, the compound of formula I is in a formulation as described herein.

In some embodiments, the formulation is administered to the ocular surface of the subject, e.g., any part of the cornea, conjunctiva, or to the cul de sac of the eye.

In some embodiments, the invention provides for the administration of the compound of formula I to a subject in need thereof in a ophthalmically compatible formulation. In some embodiments, the compound of formula I is administered to the subject one to six times a day, e.g., one, two, three, or four times a day. In some embodiments, the compound of formula I is administered to the subject for a period of at least about one month, at least about two months, or at least about three months. In some embodiments, the compound of formula I is administered to the subject for a period of at least about 12 weeks.

In some embodiments, the ocular surface pain or the chronic ocular surface pain is associated with one or more of dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, or patients recovering from neurotrophic keratitis.

In particular embodiments, the ocular surface pain or the chronic ocular surface pain is associated with dry eye disease or Sjogren's Syndrome. In some embodiments, the subject suffers from conjunctivitis, subconjunctival hemorrhage, subconjunctival scarring, conjunctival membranes, conjunctival ulceration, superficial punctate epithelial erosions, epithelial defects, lid margin ulceration, lid margin keratinization, symblepharon, ankyloblepharon, trichiasis, anterior blepharitis, punctal auto-occlusion, meibomian gland disease, corneal opacification, dry eye, districhiasis, limbal stem cell failure, or corneal vascularization.

In some embodiments, the administration of compound of formula I results in a reduction in the subject's ocular pain, compared to a placebo. In some embodiments, the reduction in the subjects ocular pain is at least about 3 when measured on the VAS score, compared to a placebo. In some embodiments, the administration results in a reduction in the subject's ocular pain of at least about 4, at least about 5, at least about 6, at least about 7, at least about 8, at least about 9, or at least about 10, when measured on the VAS score, compared to a placebo. In some embodiments, the administration results in a reduction in the subject's pain of at least about 10%, at least about 15%, at least about 20%, or at least about 25%, compared to a placebo.

In some embodiments, the administration of the compound of formula I results in a reduction in the subject's pain of at least about 2 compared to a placebo, as measured by the VAS score, about half hour after the administration, about one hour, about 2 hours, or about 2-4 hours after the administration.

In some embodiments, the reduction in pain score arises from the difference in pain scores prior to and after administration of compound I to the subject. In some embodiments, the reduction in pain score as measured by the VAS, arises from the difference in pain scores prior to and after administration of compound I to the subject. In some embodiments, the reduction in pain score occurs within about half hour after administration of compound I to the subject. In some embodiments, the reduction in pain score occurs within about 1 hour, about 2 hours, about 3 hours, about 4 hours, about 5 hours, or about 6 hours after administration of compound I to the subject.

In some embodiments, the administration of the compound of formula I results in reduced ocular hyperemia (redness of the eye), compared to placebo. In particular embodiments, the administration of the compound of formula I results in reduced grade 1, grade 2, grade 3, or grade 4 hyperemia compared to placebo.

In some embodiments, the administration results in a reduction in ocular hyperemia score of at least about 1, at least about 2, at least about 3, at least about 4, or at least about 5, on the McMonnies scale.

Thus, in some embodiments, the present invention relates to a method of treating or reducing ocular hyperemia in a subject in need thereof, comprising administering to the subject an effective amount of compound of formula I, or a pharmaceutically acceptable salt, solvate, polymorph, or co-crystal thereof. In some embodiments, the invention provides for the use of the compound of formula I, or a pharmaceutically acceptable salt, solvate, or co-crystal thereof, in the treatment of ocular hyperemia. In some embodiments, the administration results in a reduction in ocular hyperemia score of at least about 1, at least about 2, at least about 3, at least about 4, or at least about 5, on the McMonnies scale. In some embodiments, the invention provides for the administration of the compound of formula I to a subject in need thereof in a ophthalmically compatible formulation at a concentration of about 0.5% w/v to about 3.5% w/v. In some embodiments, concentrations for administration range from about 0.5% to about 3.5% w/v, about 0.5% to about 2.5% w/v, about 0.5% to about 1.5% w/v, about 0.5% to about 3.0% w/v, about 1.0% to about 2.5% w/v, about 1.5% to about 3.0% w/v, about 0.5% to about 2.5% w/v. In particular embodiments, the concentration of the compound of formula I in a formulation for topical use is about 0.5% w/v, about 1.0% w/v, about 1.5% w/v, about 2.0% w/v, about 2.5% w/v, about 3.0% w/v, or about 3.5% w/v. In some embodiments, the dose per administration per eye is from about 0.15 to about 1.15 mg, or about 0.15 mg, 0.2 mg, about 0.25 mg, 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1.0 mg, about 1.05 mg, about 1.1 mg, or about 1.15 mg. In some embodiments, the dose per administration per eye is about 0.18 mg, about 0.37 mg, about 0.55 mg, about 0.74 mg, or about 0.92 mg. In some embodiments, the total daily dose per eye is about 0.5 to about 3.5 mg, or about 0.5 mg, about 1.0 mg, about 1.5 mg, about 2.0 mg, about 2.5 mg, about 3.0 mg, or about 3.5 mg. In some embodiments, the compound of formula I is administered to the subject one to six times a day, e.g., one, two, three, or four times a day. In some embodiments, the compound of formula I is administered to the subject for a period of at least about one month, at least about two months, or at least about three months. In particular embodiments, the compound of formula I is administered in a formulation described herein.

In some embodiments, the ocular hyperemia is associated with one or more of dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, or patients recovering from neurotrophic keratitis.

In some embodiments, the ocular surface pain or chronic ocular surface pain is associated with dry eye disease. In some embodiments, the administration of the compound of formula I results in a decrease in the symptoms of dry eye disease. Dry eye disease is generally understood to be a complex, multifactorial condition characterized by inflammation of the ocular surface and lacrimal glands and reductions in the quality and/or quantity of tears. It is believed that up to 30% of dry eye disease patients suffer from ocular surface pain that may be chronic. Thus, in some embodiments, the invention results in a decrease of at least about 10%, at least about 15%, at least about 20%, or at least about 30% in the symptoms of dry eye disease, including one or more of ocular dryness, ocular discomfort, ocular hyperemia, ocular burning or stinging, grittiness or foreign body sensation, or photophobia.

In some embodiments, the invention relates to a method of treating dry eye disease in a subject in need thereof, comprising administering to the subject an effective amount of compound of formula I, or a pharmaceutically acceptable salt, solvate, polymorph, or co-crystal thereof. In some embodiments, the invention relates to a method of treating dry eye disease in a subject in need thereof, comprising administering to the subject an effective amount of compound of formula I, or a pharmaceutically acceptable salt, solvate, polymorph, or co-crystal thereof, wherein the compound of formula I is safe for administration over a period of at least 2 months, at least 3 months, at least 4 months, or at least 5 months. In particular embodiments, the invention provides for the use of the compound of formula I, or a pharmaceutically acceptable salt, solvate, or co-crystal thereof, in the treatment of dry eye disease. In some embodiments, the invention results in a decrease of at least about 10% in the symptoms of dry eye disease, including one or more of ocular dryness, ocular discomfort, ocular hyperemia, ocular burning or stinging, grittiness or foreign body sensation, or photophobia. In some embodiments, the invention provides for the administration of the compound of formula I to a subject in need thereof in a ophthalmically compatible formulation at a concentration of about 0.5% w/v to about 3.5% w/v. In some embodiments, concentrations for administration range from about 0.5% to about 3.5% w/v, about 0.5% to about 2.5% w/v, about 0.5% to about 1.5% w/v, about 0.5% to about 3.0% w/v, about 1.0% to about 2.5% w/v, about 1.5% to about 3.0% w/v, about 0.5% to about 2.5% w/v. In particular embodiments, the concentration of the compound of formula I in a formulation for topical use is about 0.5% w/v, about 1.0% w/v, about 1.5% w/v, about 2.0% w/v, about 2.5% w/v, about 3.0% w/v, or about 3.5% w/v. In some embodiments, the dose per administration per eye is from about 0.15 to about 1.15 mg, or about 0.15 mg, 0.2 mg, about 0.25 mg, 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1.0 mg, about 1.05 mg, about 1.1 mg, or about 1.15 mg. In some embodiments, the dose per administration per eye is about 0.18 mg, about 0.37 mg, about 0.55 mg, about 0.74 mg, or about 0.92 mg. In some embodiments, the total daily dose per eye is about 0.5 to about 3.5 mg, or about 0.5 mg, about 1.0 mg, about 1.5 mg, about 2.0 mg, about 2.5 mg, about 3.0 mg, or about 3.5 mg. In some embodiments, the compound of formula I is administered to the subject one to six times a day, e.g., one, two, three, or four times a day. In some embodiments, the compound of formula I is administered to the subject for a period of at least about one month, at least about two months, or at least about three months. In some embodiments, the compound of formula I is administered as a formulation described herein.

In some embodiments of the methods described herein, the administration of the compound of formula I does not result in a change (e.g., of less than 5% difference, less than 4% difference, or less than 3% difference) in one or more of best corrected visual acuity, slit-lamp biomicroscopy, dilated eye exam, blink rate, tear production, intraocular pressure or corneal staining, compared to a placebo. In some embodiments of the methods described herein, the administration of compound of formula I does not result in a delay in wound healing compared to a placebo in a patient in need thereof.

### Patient Population

In specific embodiments, a subject to be treated by methods provided herein suffers from an ocular surface disorder. Non-limiting examples of ocular surface disorders include chronic ocular surface pain (COSP), dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, or patients recovering from neurotrophic keratitis.

In certain embodiments, methods provided herein is for treating, or reducing, ocular surface pain, such as acute ocular surface pain.

In certain embodiments, methods provided herein is for treating, or reducing, ocular surface pain, such as chronic ocular surface pain (COSP). In particular aspects, COSP is characterized as persistent ocular surface pain (e.g., persistent severe ocular surface pain) that can distract from, or can interfere with, regular daily activities. In specific aspects, COSP can result in poor quality of life, and can persist for at least 1 month, at least 2 months, at least 3 months, at least 4 months, at least 5 months, or at least 6 months. In some aspects, COSP can persist for at least about 2 months or at least about 3 months. In other aspects, COSP can persist for at least 3 months or at least 4 months. In particular aspects, subject with COSP remain symptomatic despite adherence to other therapies indicated for their underlying disease (e.g., an ocular surface disorder such as dry eye disease or Sjogren's Syndrome).

In some embodiments, the subject to be treated suffers from ocular neuropathic pain (ONP). ONP is a spectrum of disorders of ocular pain that may be caused by damage or disease affecting the nerves, e.g., corneal nerves. Symptoms of ONP may include one or more of eye pain, sensitivity to light, hyperalgesia or dysesthesia (abnormal sensations) such as a sensation of dryness, stinging, or foreign body, pain from normally non-painful stimuli (allodynia). Gabapentin and other neuropathic pain medications may be used to blunt sensory nerve stimulation or the perception of nerve stimulation.

In some embodiments, the subject to be treated suffers from exposure keratopathy. EK is damage to the cornea that occurs primarily from prolonged exposure of the ocular surface to the outside environment. EK can lead to ulceration, microbial keratitis, and permanent vision loss from scarring. Patients at risk for EK include those who suffer from conditions that interfere with the ability to protect the cornea; either by incomplete eyelid closure (e.g., lagophthalmos, proptosis, lid malposition), inadequate blink reflex, inadequate blink rate (for example, caused by a neurologic disease, e.g., Parkinson disease, a neuromuscular disease) and/or decreased protective lubrication of the cornea. Symptoms of EK include foreign body sensation, burning, increased tearing, and intermittent blurry vision (from an unstable tear film), pain and photophobia. Standard treatments include the use of frequent artificial tears with nightly lubricating ointment, punctal plugs.

In some embodiments, the subject to be treated suffers from keratoconjunctivitis. Keratoconjuctivitis is an inflammatory process that involves both the conjunctiva and the cornea. Superficial inflammation of the cornea (keratitis) occurs commonly in association with viral and bacterial conjunctivitis, for example in adults. The following types of keratoconjuctivitis are distinguished based on the potential cause of inflammation:
Keratoconjunctivitis sicca is cause by the inflammation due to dryness;
Vernal keratoconjunctivitis (VKC) occurs seasonally, considered to be due to allergens;
Atopic keratoconjunctivitis is one manifestation of atopy;
Epidemic keratoconjunctivitis or adenoviral keratoconjunctivitis is caused by an adenovirus infection;
Infectious bovine keratoconjunctivitis (IBK) is a disease affecting cattle caused by the bacteria Moraxella bovis;
Pink eye in sheep and goat is mostly caused by Chlamydophila pecorum; “Superior limbic keratoconjunctivitis is thought to be caused by mechanical trauma;
Keratoconjunctivitis photoelectrica (arc eye) means inflammation caused by photoelectric UV light.

In some embodiments, the subject to be treated suffers from dry eye. The term “dry eye” as used herein, refers to inadequate tear production and/or abnormal tear composition. Dry eye syndrome disease (DEDS), also known as dry eye syndrome, keratoconjunctivitis sicca or keratitis sicca, or tear dysfunction syndrome, or burning eye syndrome results from deficiency of any of the tear film layers. Dry eye is a multifactorial disease of the tears and ocular surface that results in symptoms of discomfort, visual disturbance, and tear ¬film instability with potential damage to the ocular surfaceocular surface characterized by loss of homeostasis of the tear film, and accompanied by ocular symptoms, in which tear film instability and hyperosmolarity, ocular surface inflammation and damage, and neuro-sensory abnormalities play etiological roles (Craig J P, et al., The Ocular Surface 2017; 15:276-83). It may be accompanied by increased osmolarity of the tear film and inflammation of the ocular surface. Dry eye disorder may range from mild to moderate to severe forms. Symptoms of dry eye syndrome disease include gritty, foreign body sensations, burning, photophobia, and decreased visual acuity, tearing, stinging, itching, sandy or gritty feeling, discharge, frequent blinking, mattering or caking of the eyelashes (usually worse upon waking), redness, blurry or fluctuating vision (made worse when reading, computer, watching television, driving, or playing video games), light-sensitivity, eye pain and/or headache, heavy eye lids, eye fatigue. Causes of dry eye disease include, but are not limited to, the following: idiopathic, congenital alacrima, xerophthalmia, lacrimal gland ablation, and sensory denervation; collagen vascular diseases, including rheumatoid arthritis, Wegener's granulomatosis, and systemic lupus erythematosus; Sjogren's Syndrome and autoimmune diseases associated with Sjogren's syndrome; abnormalities of the lipid tear layer caused by blepharitis or rosacea; abnormalities of the mucin tear layer caused by vitamin A deficiency; trachoma, diphtheric keratoconjunctivitis; mucocutaneous disorders; aging; menopause; and diabetes. Dry eye signs and/or symptoms as defined herein may also be provoked by other circumstances, including but not limited to the following: prolonged visual tasking; working on a computer; being in a dry environment; warm or cold wind or air flow; seasonal changes; ocular irritation; contact lenses, LASIK and other refractive surgeries; fatigue; and medications such as isotretinoin, sedatives, diuretics, tricyclic antidepressants, antihypertensives, oral contraceptives, antihistamines, nasal decongestants, beta-blockers, phenothiazines, atropine, and pain relieving opiates such as morphine.

Diagnostic testing for dry eye includes evaluation of cornea sensation (corneal hyperesthesia and/or reduced sensation may be present in severe and chronic dry eye disease) using, for example, a cotton tip applicator or more precisely with a Cochet-Bonnet esthesiometer; measuring tear break up time using, for example, a fluorescein-impregnated strip wet with non-preserved saline solution or more objective computerized methods without the need for fluorescein instillation; performing oculr surface staining, e.g., fluorescein sodium, rose bengal, lissamine green; performing Schirmer test (relatively insensitive for patients with mild dry eye), testing delayed tear clearance; tear meniscus height; measuring level of MMP-9 (MMP-9 has been shown to be elevated in the tears of patient with dry eye disease, and levels correlate with examination findings in patients with moderate to severe dry eye); measuring tear osmolarity and tear film interferometry; performing Sjo test (detection of SS-A (anti-Ro) and SS-B (anti-La) autoantibodies in serum, salivary gland protein 1 (SP-1), carbonic anhydrase 6 (CA6), and parotid secretory protein (PSP). SP-1, CA6, and PSP).

Artificial tears, lubricating ointments, corticosteroids (e.g., loteprednol 0.5% eyedrops four times a day) are used as an initial treatment. Prescription medicines include cyclosporine, lifitegrast, diquafosol, rebamepide, corticosteroids (e.g., loteprednol 0.5% eyedrops four times a day).

The term “tear film dysfunction” refers to a state when the tear film breaks down in different places on the cornea and conjunctiva, leading not only to symptoms of irritation, but also to unstable and intermittently changing vision. For example, dry eye syndrome disease is characterized by tear film dysfunction. The symptoms of tear film dysfunction include tearing, burning, stinging, itching, sandy or gritty feeling, scratchy or foreign-body sensation, discharge, frequent blinking, mattering or caking of the eyelashes (usually worse upon waking), redness, blurry or fluctuating vision (made worse when reading, computer, watching television, driving, or playing video games), light-sensitivity, eye pain and/or headache, heavy eye lids, eye fatigue.

Adenoviral keratoconjunctivitis, also known as Keratoconjunctivitis epidemica is a common and highly contagious viral infection of the eye. The clinical course of Adenoviral keratoconjunctivitis is divided into an acute phase with conjunctival inflammation of varying intensity with or without corneal involvement and a chronic phase with corneal opacities.

Vernal keratoconjunctivitis (VKC) is an atopic condition of the external ocular surface characterized by symptoms consisting of severe itching, photophobia, foreign body sensation, mucous discharge (often described as “ropy”), blepharospasm, and blurring of vision (Buckley, R. J., Int Ophthalmol C/in, 1988 28(4): p. 303-8; Kumar, S., Acta Ophthalmologica, 2009. 87(2): p. 133-147). It is typically bilateral but may be asymmetric in nature. It characteristically affects young males in hot dry climates in a seasonal manner; in 23% of patients may have a perennial form (Kumar, S., Acta Ophthalmologica, 2009. 87(2): p. 133-147; Bonini, S., et al., Ophthalmology, 2000. 107(6): p. 1157-63).

The signs of VKC can be divided into conjunctival, limbal and corneal signs:
Conjunctival signs include diffuse conjunctival injection and upper tarsal giant papillae that are discrete >1 mm in diameter;
Limbal signs include thickening and opacification of the limbal conjunctiva as well as gelatinous appearing and sometime confluent limbal papillae. Peri-limbal Homer-Trantas dots are focal white limbal dots consisting of degenerated epithelial cells and eosinophils (Buckley, R. J., Int Ophthalmol Clin, 1988. 28(4): p. 303-8);
Corneal signs vary according to the severity of the disease process and include macro-erosions, comal ulcers and scars (Buckley, R. J., Int Ophthalmol Clin, 1988. 28(4): p. 303-8).

Active VKC patients (defined as moderate to severe ocular discomfort including photophobia, papillae on the upper tarsal conjunctiva, or limbal Horner-Trantas dots clearly recognizable at the time of the examination) showed significantly increased symptoms and signs of ocular surface disease. Inactive VKC patients (defined as no symptoms or mild discomfort, and absence of corneal abnormalities at the time of the examination) showed increased photophobia, conjunctival lissamine green staining and Schirmer test values, and reduced fluorescein break-up time (BUT) and corneal sensitivity. This syndrome seems to affect the ocular surface in all phases (active and quiescent), determining abnormalities in tear film stability, epithelial cells integrity, and corneal nerves function (Villani E. et al., Medicine (Baltimore). 2015 October; 94(42): e1648).

The following factors are thought to play a role in VKC: IgE mediate reaction via mast cell release; activated eosinophils, mononuclear cells and neutrophils as well as the CD4 T-helper-2 driven type IV hypersensitivity with immunomodulators such as IL-4, IL-5, and bFGF (Buckley, R. J., Int Ophthalmol Clin, 1988. 28(4): p. 303-8; Kumar, S., Acta Ophthalmologica, 2009. 87(2): p. 133-147; La Rosa, M., et al., Ital J Pediatr, 2013. 39: p. 18).

Treatment consists of cool compresses and lid scrubs, saline eyedrops, which may help to relieve symptoms, along with topical antihistamines, nonsteroidal anti-inflammatory drugs or corticosteroids, e.g., low-absorptions corticosteroids (fluoromethelone, loteprednol, remexolone, etc.), optical mast cell stabilizers (cromolyn sodium, nedocromil sodium, and lodoxamide), topical cyclosporin-A, or tacrolimus. See e.g., Oray, M. and E. Toker, Cornea, 2013. 32(8): p. 1149-54: Vichyanond, P. and P. Kosrirukvongs, Curr Allergy Asthma Rep, 2013. 13(3): p. 308-14; Barot, R K et al., J Clin Diagn Res. 2016 June; 10(6):NC05-9; Wan Q et al., Ophthalmic Res. 2018; 59(3):126-134.

Atopic keratoconjunctivitis (AKC) typically has an older age of onset in the 2nd to 5th decade, as opposed to onset prior to age 10 with VKC. Conjunctival involvement is classically on the upper tarsus in VKC and on the lower tarsus in AKC. AKC is typically more chronic in nature and more commonly results in scarring of the cornea and conjunctival cicatrization.

Sjogren's Syndrome (Sjogren's syndrome associated with dry eye) is a chronic inflammatory disorder characterized by exocrine gland dysfunction including the salivary and lacrimal glands that in many cases results in a severe dry eye. Primary symptoms are dry eyes (keratitis sicca or keratoconjunctivitis sicca) and dry mouth (xerostomia). Severe dry eyes can cause corneal pain, corneal scarring, ulceration, infection, and even perforation. The differential diagnosis includes conditions such as adult blepharitis, dry eye disease, and juvenile idiopathic arthritis uveitis, as well keratopathies, e.g., superficial punctate, filamentary, neurotrophic, exposure). Treatment of Sjogren's syndrome is aimed at maintaining the integrity of the tear film through preservation, augmentation, and/or replacement of the deficient tear secretion. Treatment of Sjogren's syndrome thus includes artificial tears and lubricating ointments; autologous serum eyedrops; oral omega-6 essential fatty acids; fluid-ventilated, gas permeable scleral lenses; topical corticosteroids; punctal occlusion to decrease tear drainage; a small lateral tarsorrhaphy; humidification of the environment; hydrophilic bandage lenses; bromhexine and 3-isobutyl 1-methylxanthine (IBMX) (augmentation of tear production/secretion); agents to stimulate muscarinic receptors (pilocarpine and cevimeline); immunosuppressive agents, e.g., methotrexate, antimalarials, cyclophosphamide, leflunomide, or tumor necrosis factor (TNF), e.g., infliximab, a monoclonal antibody to TNF-alpha; Cyclosporin A; the bandage contact lens.

Steven-Johnson's syndrome (SJS) is a dermatologic emergency or a type of severe skin reaction characterized by the presence of epidermal and mucosal bullous lesions involving less than 10% of the total body surface area. Early symptoms of SJS include fever and flu-like symptoms, which may precede or occur concurrently with the development of a macular rash involving the trunk and face. As the disease progresses, the macular rash coalesces, the involved areas develop bullae, and the epidermal layer eventually sloughs off. During the acute phase of SJS-TEN, 80% of patients will have ocular involvement.

The constellation of high fever (>102.2), malaise, arthralgia, a macular rash involving the trunk, neck and face, and recent history of new medication exposure or recently increased dosage of an existing medication are indicators used for diagnosis of SJS. A skin biopsy of an effected area can be performed for a confirmation of the diagnosis. Granulysin can be used as a marker for the diagnosis of SJS. The concentration of granulysin within bullous fluid correlates with the severity of the acute phase of SJS (Chung W H, et al. Nat Med. 2008; 14(12):1343-50).

Ocular manifestations in SJS include conjunctivitis, subconjunctival hemorrhage, subconjunctival scarring, conjunctival membranes, conjunctival ulceration, superficial punctate epithelial erosions, epithelial defects, lid margin ulceration, lid margin keratinization, symblepharon, ankyloblepharon, trichiasis, anterior blepharitis, punctal auto-occlusion, meibomian gland disease, corneal opacification, dry eye, districhiasis, limbal stem cell failure, corneal vascularization. Eye treatment in SJS consists of saline eyedrops, preservative-free artificial tears and ointments to provide adequate lubrication and reduce epithelial injury. Patients with any corneal or conjunctival epithelial defects are treated with prophylactic topical antibiotics, e.g., a fourth generation fluoroquinolone. Patients having mild or moderate ocular involvement (less than one-third lid margin involvement, conjunctival defects less than 1 cm at greatest diameter, and no corneal epithelial defects) are typically treated with topical moxifloxacin 0.5% four times a day, cyclosporine 0.05% twice daily, and topical steroids (prednisolone acetate 1% four to eight times a day or dexamethasone 0.10% twice daily). Patients having severe or extremely severe ocular involvement (greater than one-third lid margin involvement, conjunctival defects greater than 1 cm, and corneal epithelial defects) undergo an amniotic membrane (AM) grafting in addition to the treatments listed above.

In some embodiments, the subject to be treated suffers from corneal epithelipathy. Corneal epitheliopathy is a disease involving corneal epithelium, e.g., manifested in altered corneal epithelial barrier function.

In some embodiments, the subject to be treated suffers from corneal neuropathy or corneal neuralgia. Corneal neuropathy or corneal neuralgia is a disorder associated with corneal pain caused by the damaged nerve fibers in the cornea, the sensory fibers. One of the examples of corneal neuropathy is a LASIK induced corneal neuropathy. Corneal neuropathy generally could be identified and diagnosed through dry eye investigations. Though the causes and risk factors are unclear yet, patients with dry eye-like symptoms, increased corneal sensitivity and changes of corneal nerve morphology, but no signs of dryness may suffer from corneal neuropathy.

In some embodiments, the subject to be treated suffers from ocular surface disease or disorder. The term “ocular surface diseases” or “ocular surface disorders” encompasses disease entities as well as related symptoms that result from a variety of abnormalities, including abnormal lid anatomy or function, abnormal or altered tear production or composition, and related subclinical signs. Many diseases can cause ocular surface disorders. Patients with ocular surface disorders may exhibit clinical signs common to several diseases, and include chronic punctate keratopathy, filamentary keratopathy, recurrent corneal erosion, bacterial conjunctivitis, culture-negative conjunctivitis, cicatrising (scarring) conjunctivitis, persistent epithelial defect, infectious keratitis, corneal melt and ocular surface failure. The most common ocular surface disorders stem from tear-film abnormalities and/or lid-gland dysfuntion (“blepharitis”).

In some embodiments, the subject to be treated suffers from neurotrophic keratitis or neurotrophic keratopathy. Neurotrophic keratitis or neurootrophic keratopathy (NK) is a corneal degenerative disease characterized by a reduction or absence of corneal sensitivity. In NK, corneal innervation by trigeminal nerve is impaired. Since corneal sensory innervation is impaired in NK, patients do not commonly complain of ocular surface symptoms. However, blurred vision can be reported due to irregular epithelium or epithelial defects (PED), scarring, or edema. NK is usually graded in three different stages in accordance to the “Mackie classification”. Stage II NK is defined by a recurrent or persistent epithelial defects, most commonly in the superior half of the cornea. One of the treatments that may be used in Stage II NK includes topical Nerve Growth Factor. Patients typically experience pain during treatment with NGF due to reforming of the nerves.

In some embodiments, the subject to be treated suffers from blepharitis. Blepharitis is an inflammatory condition of the eyelid margin, which can lead to permanent alterations in the eyelid margin or vision loss from superficial keratopathy, corneal neovascularization, and ulceration. According to anatomic location, blepharitis can be divided into anterior and posterior. Anterior blepharitis affects the eyelid skin, base of the eyelashes, and the eyelash follicles and includes the traditional classifications of staphylococcal and seborrheic blepharitis. Posterior blepharitis affects the meibomian glands and gland orifices, the primary cause being meibomian gland dysfunction. Symptoms of chronic blepharitis may include redness, burning sensation, irritation, tearing, eyelid crusting and sticking, and visual problems such as photophobia and blurred vision. Long-term management of symptoms may include daily eyelid cleansing routines and the use of therapeutic agents that reduce infection and inflammation. Treatment includes topical or systemic antibiotics e.g., bacitracin or erythromycin; oral antibiotics, e.g., tetracyclines (tetracycline, doxycycline, minocycline) or macrolides (erythromycin, azithromycin); topical steroids, e.g., corticosteroid, e.g., loteprednol etabonate, fluorometholone; topical combinations of an antibiotic and corticosteroid such as tobramycin/dexamethasone or tobramycin/loteprednol; topical cyclosporine 0.05%.

In some embodiments, the subject to be treated suffers from Meibomian gland dysfunction. The meibomian gland is a holocrine type of exocrine gland, at the rim of the eyelid inside the tarsal plate, responsible for the supply of meibum, an oily substance that prevents evaporation of the eye's tear film. Meibomian gland dysfunction (MGD), also known as meibomitis, posterior blepharitis or inflammation of the meibomian glands, is a chronic, diffuse abnormality of the meibomian glands, commonly characterized by terminal duct obstruction and/or qualitative/quantitative changes in the glandular secretion (Nelson J D, et al., Invest Ophthalmol Vis Sci 2011; 52:1930-7). It may result in alteration of the tear film, symptoms of eye irritation, clinically apparent inflammation, and ocular surface disease. MGD often causes dry eye, and may contribute to blepharitis. In some cases topical steroids and topical/oral antibiotics are also prescribed reduce inflammation. Intense pulsed light (IPL) treatments or other mechanical treatments that apply heat and pressure to express the glands (eg, LipiFlow) have also been shown to reduce inflammation and improve the gland function in patients.

In some embodiments, the subject to be treated suffers from graft-versus-host disease. Graft-versus-host disease (GVHD) is an inflammatory disease that is unique to allogeneic transplantation. It is an attack by transplanted leukocytes against the recipient's tissues that can occur even if the donor and recipient are HLA-identical. Acute graft-versus-host disease typically occurs in the first 3 months after transplantation and may involve the skin, intestine, or the liver. Corticosteroids such as prednisone are a standard treatment. Chronic graft-versus-host disease may also develop after allogeneic transplant and is the major source of late complications. In addition to inflammation, chronic graft-versus-host disease may lead to the development of fibrosis, or scar tissue, similar to scleroderma or other autoimmune diseases and may cause functional disability, and the need for prolonged immunosuppressive therapy.

In some embodiments, the subject to be treated suffers from ocular graft versus host disease. GVHD occurs in patients who have undergone allogenic hematological stem cell transplantation. It can occur in patients who have acute or chronic GVHD, though it is more common in patients with the chronic form. Approximately 40-90% of patients with chronic GVHD will develop ocular symptoms. Ocular manifestations can include moderate to severe keratoconjuncitvitis sicca, bilateral marginal keratitis, anterior uveitis, corneal ulceration or neovascularization. Treatment includes topical lubricants including preservative free artificial tears, autologous serum tears and other topical and systemic immunosuppressive treatments; systemic steroids; topical cyclosporine 0.5%.

### EXAMPLES

The following examples are included to demonstrate non-limiting embodiments of the present invention.

### General Test Conditions

The following procedures were employed under each test condition.

| TGA method | |
|---|---|
| Instrument | TA Discovery |
| Temperature range | Room temperature to 300° C. |
| Scan rate | 10 K/min |
| Nitrogen flow | 20 mL/min |
| Sample mass | Approximately 2-10 mg |
| DSC method | |
| Instrument | TA Discovery |
| Temperature range | 0° C. to 250° C. or 300° C. |
| Scan rate | 10 K/min |
| Nitrogen flow | 50 mL/min |
| Sample mass | Approximately 2 mg |

XRPD method 1 & 2

| | |
|---|---|
| Instrument | Bruker D8 Advance |
| Detector | LynxEye (1D mode), open angle: 2.948° |
| Radiation | CuK_{α} (0.15418 nm) |
| Monochromator | Ni-filter |
| X-ray generator | 40 kV, 40 mA |
| power | |
| Step size | 0.0164° or 0.0410° (2 theta) |
| Time per step | 0.3 s |
| Scan range | 2º-40° 2 theta |
| Scan time | 768 s or 279 s |
| Slits | Primary fixed illuminated sample size: 10 mm, |
| | secondary: open angle: 2.2°, axial soller: 2.5° |

One of ordinary skill in the art will appreciate that an X-ray diffraction pattern may be obtained with a measurement error that is dependent upon the measurement conditions employed. In particular, it is generally known that intensities in a X-ray diffraction pattern may fluctuate depending upon measurement conditions employed. It should be further understood that relative intensities may also vary depending upon experimental conditions and wavelength of X-ray radiation used. The agreement in the 2-theta-diffraction angles between specimen and reference is within 0.2° for the same crystal form and such degree of measurement error should be taken into account as pertaining to the aforementioned diffraction angles. Consequently, it is to be understood that the crystal forms of the instant invention are not limited to the crystal forms that provide X-ray diffraction patterns completely identical to the X-ray diffraction patterns depicted in the accompanying Figures disclosed herein. Any crystal forms that provide X-ray diffraction patterns substantially identical to those disclosed in the accompanying Figures fall within the scope of the present invention. The ability to ascertain substantial identities of X-ray diffraction patterns is within the purview of one of ordinary skill in the art.

| DVS | |
|---|---|
| Instrument | Advantage or Intrinsic |
| Sample mass | approximately 10 mg |
| Temperature | 25° C. or 40° C. |
| Program | 40-0-95-0-40 (% RH) with dm/dt 0.002%/min |

NMR

| | |
|---|---|
| Instrument | Bruker ASCEND 400 MHZ |
| Probe | 5mm PABBO BB-1H/D Z-GRD Z108618/0226 |
| Temperature | 295.7 K |
| Relaxation delay | 1 second |

### Example 1. Preparation of Compound of Formula I

### Synthesis of (S)-1-(6-fluoro-5-((((1r,3S)-3-(4-fluoro-3-(trifluoromethyl)phenoxy)cyclobutyl)amino)methyl)isoquinolin-8-yl)ethane-1,2-diol (or trans-(S)-1-(6-fluoro-5-(((3-(4-fluoro-3-(trifluoromethyl)phenoxy)cyclobutyl)amino)methyl)isoquinolin-8-yl)ethane-1,2-diol) and (R)-1-(6-fluoro-5-((((1r,3R)-3-(4-fluoro-3-(trifluoromethyl)phenoxy)cyclobutyl)amino)methyl)isoquinolin-8-yl)ethane-1,2-diol (or trans-(R)-1-(6-fluoro-5-(((3-(4-fluoro-3-(trifluoromethyl)phenoxy)cyclobutyl)amino)methyl)isoquinolin-8-yl)ethane-1,2-diol)

### Step 1.1: Synthesis of 6-fluoro-8-(oxiran-2-yl)isoquinoline

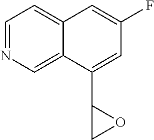 [SMILES: Fc1cc(C2CO2)c2cnccc2c1]

To the solution of NaH (1.0 g, 41.45 mmol) and anhydrous DMSO (40 mL), trimethylsulfoxonium iodide (8.3 g, 37.68 mmol) was added at rt and stirred for 30 min. Then 6-fluoroisoquinoline-8-carbaldehyde (Step 6.5, 3.3 g, 18.84 mmol), dissolved in DMSO (20 mL), was added dropwise at rt. After 5 min, the reaction was quenched with ice-water and extracted with EtOAc 3×'s. The combined organic portion was washed with brine, dried over anhydrous Na₂SO₄, filtered and concentrated in vacuo. The residue was purified by flash chromatography (12 g SiliCycle column, 0-20% EtOAc in Hexane elution) to provide 6-fluoro-8-(oxiran-2-yl)isoquinoline (2.3 g, 64%). MS (ESI+) [Method 6A]: m/z 190.1 (M+H); Rt 0.79 min. ¹H NMR (600 MHz, CDCl₃) δ 9.55 (s, 1H), 8.58 (d, J=5.4 Hz, 1H), 7.65 (d, J=5.4 Hz, 1H), 7.37 (d, J=9.0 Hz, 2H), 4.60-4.59 (m, 1H), 3.37-3.35 (m, 1H), 2.82-2.80 (m, 1H).

### Step 1.2: Synthesis of 1-(6-fluoroisoquinolin-8-yl)ethane-1,2-diol

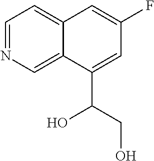 [SMILES: OCC(O)c1cc(F)cc2ccncc12]

To the solution of 6-fluoro-8-(oxiran-2-yl)isoquinoline (2.1 g, 11.11 mmol) in THF-H₂O (12 mL, 2:1 v/v), H₂SO₄ (5 mL) was added dropwise at rt and stirred at 60° C. for 16 h. The reaction mixture was basified with saturated NaHCO₃ solution and extracted with EtOAc twice. The combined organic portion was washed with brine, dried over anhydrous Na₂SO₄, filtered and concentrated in vacuo. The residue was purified by flash chromatography (12 g SiliCycle column, 0-5% MeOH in CH₂Cl₂ elution) to provide 1-(6-fluoroisoquinolin-8-yl)ethane-1,2-diol (1.6 g, 69%). MS (ESI+) [Method 4A]: m/z 208.3 (M+H); Rt 0.40 min. ¹H NMR (600 MHz, CDCl₃) δ 9.49 (s, 1H), 8.49 (d, J=6.0 Hz, 1H), 7.67-7.63 (m, 2H), 7.35 (dd, J=8.4, 1.8 Hz, 1H), 4.13-4.10 (m, 1H), 4.06 (dd, J=12.6, 3.6 Hz, 1H), 3.76 (dd, J=11.4, 3.6 Hz, 1H).

### Step 1.3: Synthesis of 6-fluoro-8-(2,2,3,3,8,8,9,9-octamethyl-4,7-dioxa-3,8-disiladecan-5-yl)isoquinoline

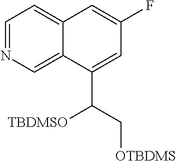 [SMILES: CC(C)(C)[Si](C)(C)OCC(O[Si](C)(C)C(C)(C)C)c1cc(F)cc2ccncc12]

To the solution of 1-(6-fluoroisoquinolin-8-yl)ethane-1,2-diol (1.5 g, 7.24 mmol) and imidazole (3.4 g, 50.68 mmol) in DMF (15 mL), TBDMS-Cl (5.4 g, 36.17 mmol) was added portion wise at 0° C. and stirred at rt for 16 h. Then the reaction mixture was diluted with water and extracted 3× with EtOAc. The combined organic portion was washed with brine, dried over anhydrous Na₂SO₄, filtered and concentrated in vacuo. The residue was purified by flash chromatography (12 g SiliCycle column, 0-10% EtOAc in Hexane elution) to afford 6-fluoro-8-(2,2,3,3,8,8,9,9-octamethyl-4,7-dioxa-3,8-disiladecan-5-yl)isoquinoline (2.7 g, 85%). MS (ESI+) [Method 6A]: m/z 436.3 (M+H); Rt 2.15 min. ¹H NMR (600 MHz, CDCl₃) δ 9.59 (s, 1H), 8.52 (d, J=4.8 Hz, 1H), 7.61 (d, J=5.4 Hz, 1H), 7.55 (dd, J=10.2, 1.8 Hz, 1H), 7.33 (dd, J=9.0, 2.4 Hz, 1H), 5.54 (d, J=6.0 Hz, 1H), 3.87-3.85 (m, 1H), 3.77-3.74 (m, 1H), 0.92 (s, 9H), 0.90 (s, 9H), 0.13 (s, 6H), 0.09 (s, 6H).

### Step 1.4: Synthesis of 6-fluoro-8-(2,2,3,3,8,8,9,9-octamethyl-4,7-dioxa-3,8-disiladecan-5-yl)isoquinoline-5-carbaldehyde

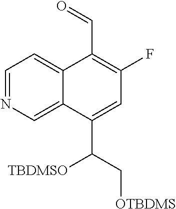 [SMILES: CC(C)(C)[Si](C)(C)OCC(O[Si](C)(C)C(C)(C)C)c1cc(F)c(C=O)c2ccncc12]

The title compound was prepared according to the procedure in Step 6.8. The residue was purified by flash chromatography (40 g SiliCycle column, 0-15% EtOAc in Hexane elution) to provide 6-fluoro-8-(2,2,3,3,8,8,9,9-octamethyl-4,7-dioxa-3,8-disiladecan-5-yl)isoquinoline-5-carbaldehyde (2.0 g, 62%). MS (ESI+) [Method 4A]: m/z 464.4 (M+H); Rt 1.77 min. ¹H NMR (600 MHz, CDCl₃) δ 9.57 (s, 1H), 8.50 (d, J=5.4 Hz, 1H), 7.60 (d, J=5.4 Hz, 1H), 7.54 (dd, J=7.8, 2.4 Hz, 1H), 7.32 (dd, J=9.0, 2.4 Hz, 1H), 5.53 (d, J=6.0 Hz, 1H), 3.87-3.84 (m, 1H), 3.76-3.73 (m, 1H), 0.89 (s, 9H), 0.75 (s, 9H), 0.12 (s, 6H), −0.05 (s, 6H).

### Step 1.5: Synthesis of (1r,3r)-3-(4-fluoro-3-(trifluoromethyl)phenoxy)-N-((6-fluoro-8-(2,2,3,3,8,8,9,9-octamethyl-4,7-dioxa-3,8-disiladecan-5-yl)isoquinolin-5-yl)methyl)cyclobutan-1-amine

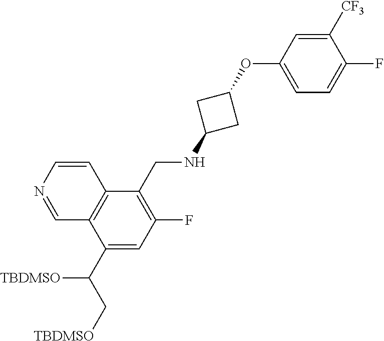 [SMILES: CC(C)(C)[Si](C)(C)OCC(O[Si](C)(C)C(C)(C)C)c1cc(F)c(CN[C@H]2C[C@H](Oc3ccc(F)c(C(F)(F)F)c3)C2)c2ccncc12]

The title compound was synthesized following the procedure as described in step 1.4, using (1r,3r)-3-(4-fluoro-3-(trifluoromethyl)phenoxy)cyclobutan-1-amine, HCl (Step 1.3, 1.0 g, 3.50 mmol) and 6-fluoro-8-(2,2,3,3,8,8,9,9-octamethyl-4,7-dioxa-3,8-disiladecan-5-yl)isoquinoline-5-carbaldehyde (1.46 g, 3.15 mmol). The crude was purified by flash chromatography (24 g SiliCycle column, 0-5% MeOH in CHCl₃ elution) to provide (1r,3r)-3-(4-fluoro-3-(trifluoromethyl)phenoxy)-N-((6-fluoro-8-(2,2,3,3,8,8,9,9-octamethyl-4,7-dioxa-3,8-disiladecan-5-yl)isoquinolin-5-yl)methyl)cyclobutan-1-amine_(1.5 g, 62%). MS (ESI+) [Method 6A]: m/z 697.3 (M+H); Rt 1.63 min.

### Step 1.6: Synthesis of (S)-1-(6-fluoro-5-((((1r,3S)-3-(4-fluoro-3-(trifluoromethyl)phenoxy)cyclobutyl)amino)methyl)isoquinolin-8-yl)ethane-1,2-diol and (R)-1-(6-fluoro-5-((((1r,3R)-3-(4-fluoro-3-(trifluoromethyl)phenoxy)cyclobutyl)amino)methyl)isoquinolin-8-yl)ethane-1,2-diol

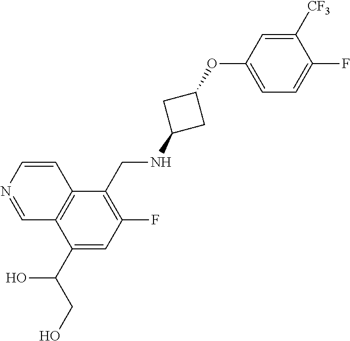 [SMILES: OCC(O)c1cc(F)c(CN[C@H]2C[C@H](Oc3ccc(F)c(C(F)(F)F)c3)C2)c2ccncc12]

To the solution (1r,3r)-3-(4-fluoro-3-(trifluoromethyl)phenoxy)-N-((6-fluoro-8-(2,2,3,3,8,8,9,9-octamethyl-4,7-dioxa-3,8-disiladecan-5-yl)isoquinolin-5-yl)methyl)cyclobutan-1-amine_(1.5 g, 2.15 mmol) in THF (25 mL), TBAF solution (1M in THF) (5.4 mL, 5.38 mmol) was added dropwise as 0° C. and stirred for 2 h. Reaction mixture was diluted with water and extracted with EtOAc twice. The combined organic portion was washed with a brine solution, dried over anhydrous Na₂SO₄, filtered and concentrated in vacuo. The residue was purified by flash chromatography (24 g SiliCycle column, 0-10% MeOH in CH₂Cl₂ elution) to afford 1-(6-fluoro-5-((((1r,3r)-3-(4-fluoro-3-(trifluoromethyl)phenoxy)cyclobutyl)amino)methyl)isoquinolin-8-yl)ethane-1,2-diol (1.0 g, 98%). MS (ESI+) [Method 6A]: m/z 469.2 (M+H); Rt 1.29 min. ¹H NMR (400 MHz, CD₃OD) δ 9.57 (s, 1H), 8.52 (d, J=6.0 Hz, 1H), 8.12 (d, J=5.6 Hz, 1H), 7.67 (d, J=10.6 Hz, 1H), 7.22 (t, J=9.6 Hz, 1H), 7.05-7.01 (m, 2H), 5.58-5.56 (m, 1H), 4.85-4.82 (m, 1H), 4.17 (d, J=1.6 Hz, 2H), 3.86-3.82 (m, 1H), 3.78-3.74 (m, 1H), 3.60-3.57 (m, 1H), 2.36-2.33 (m, 4H).

Chiral prep-HPLC (Column: CHIRALPAK IG (250 mm×20 mm); Mobile Phase: Hexane and IPA:MeOH (1:1); Isocratic: 60/40; Flow: 15 mL/min) of the racemate provided (S)-1-(6-fluoro-5-((((1r,3S)-3-(4-fluoro-3-(trifluoromethyl)phenoxy)cyclobutyl)amino)methyl)isoquinolin-8-yl)ethane-1,2-diol as a white solid Peak 1 (395 mg, 40%): Chiral HPLC: 99% (Rf 7.840 min; Column: CHIRALPAK-IG (150 mm×4.6 mm), 5.0μ; Mobile phase: n-Hexane and EtOH; Isocratic: 80/20; Flow: 1 mL/min). MS (ESI+) [Method 1A]: m/z 469.2 (M+H); Rt 1.29 min. ¹H NMR (400 MHz, CD₃OD) δ 9.57 (s, 1H), 8.53 (d, J=6.0 Hz, 1H), 8.13 (d, J=5.6 Hz, 1H), 7.68 (d, J=10.6 Hz, 1H), 7.23 (t, J=9.6 Hz, 1H), 7.06-7.01 (m, 2H), 5.59-5.56 (m, 1H), 4.85-4.82 (m, 1H), 4.19 (s, 2H), 3.87-3.83 (m, 1H), 3.78-3.74 (m, 1H), 3.60-3.57 (m, 1H), 2.37-2.34 (m, 4H); and (R)-1-(6-fluoro-5-((((1r,3R)-3-(4-fluoro-3-(trifluoromethyl)phenoxy)cyclobutyl)amino)methyl)isoquinolin-8-yl)ethane-1,2-diol as a white solid Peak 2 (345 mg, 35%) Chiral HPLC: 97% (Rf 17.481 min; Column: CHIRALPAK-IG (150 mm×4.6 mm), 5.0μ; Mobile phase: n-Hexane and EtOH; Isocratic: 80/20; Flow: 1 mL/min). MS (ESI+) [Method 3A]: m/z 469.0 (M+H); Rt 1.25 min. ¹H NMR (400 MHz, CD₃OD) δ 9.57 (s, 1H), 8.53 (d, J=6.0 Hz, 1H), 8.13 (d, J=5.6 Hz, 1H), 7.68 (d, J=10.6 Hz, 1H), 7.23 (t, J=9.6 Hz, 1H), 7.06-7.01 (m, 2H), 5.59-5.56 (m, 1H), 4.85-4.82 (m, 1H), 4.18 (d, J=1.2 Hz, 2H), 3.87-3.83 (m, 1H), 3.78-3.74 (m, 1H), 3.60-3.57 (m, 1H), 2.37-2.34 (m, 4H). Example 1 was isolated in amorphous form.

### Example 2. Crystalline Form A

Compound I was prepared as described in Example 1. Crystalline Form A was obtained from the isomeric form shown below:

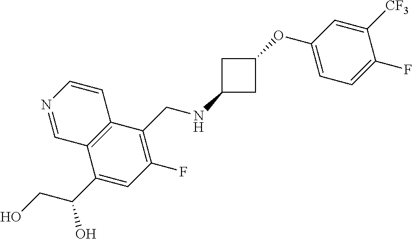 [SMILES: OC[C@@H](O)c1cc(F)c(CN[C@H]2C[C@H](Oc3ccc(F)c(C(F)(F)F)c3)C2)c2ccncc12]

The starting material for the polymorphic studies was recrystallized from acetonitrile and washed with water. About 28 g of this material was suspended in 500 ml acetonitrile and heated to 55° C. to obtain a clear solution. The solution was gradually cooled to 50° C. within 1 h and kept isothermally for a further 3 h. A small amount of solid precipitate formed and stuck to stirrer paddle. The mixture was further cooled to 25° C. within 5 h and kept at 50° C. for another 3 h, then cooled to 10° C. and kept for 5 h. The resulting solid was isolated via suction filtration, dried at 50° C. under vacuum for 4 h. A light yellow solid was obtained with a yield of about 70%.

About 13.8 g of Form A was suspended in 700 mL water and stirred at 25° C. for 6 h. The mixture was filtered and the solid re-suspended in 750 mL water. The process was repeated four times. Residual chloride was checked by ion chromatography. The solid was collected and dried at 50° C. under vacuum for 5 h. 12.8 g off-white solid was obtained with a yield of 92.7% (purity: 99.6%).

Manufacture of Form A on kg scale: A reactor was charged with 2.8 kg Compound I, 10.8 kg ethanol and 8.54 kg of water and heated to 53° C. until a clear solution was obtained. The solution was filtered through a 0.45 m filter cloth and transferred to a crystallizer where the solution was cooled down to 40° C.. 17.1 g of Form A seeds were introduced to the system and the suspension was held at 40° C. for 4 h. 10.7 kg water was introduced over 4 h and the suspension was then held for 3 h. The suspension was cooled down to 20° C. for 3 h and then held at 20° C. for 4 h. The suspension was subsequently filtered and washed with a mixture of 3.8 kg ethanol and 1.5 kg water. The wet cake was then dried at 60° C. under vacuum for 12 h to yield 2.64 kg of crystalline Form A of Compound I.

Preparation of Form A from solutions at 25° C.: About 100 mg of compound I was equilibrated with 0.5 ml of the solvent at 25° C. for 28 days under agitation. The solution was filtered and dried for 10 minutes in air. Precipitation of Compound I under these conditions in a variety of solvents yielded Form A.

TABLE 1
Crystallization of Compound I from solution at 25 ° C.

| Solvent | XRPD | Comments |
|---|---|---|
| Acetone | Form A | Gel to solid |
| Actonitrile | Form A | Plate-like particles |
| Dichloromethane | Form A | |
| 1,4-Dioxane | // | Gel-like |
| Ethanol | // | Gel-like |
| Ethyl acetate | Form A | |
| Isopropyl acetate | Form A | |
| Methanol | // | Gel-like |
| 2-Methyl-2-butanol | Form A | Gel to solid |
| MTBE | Form A | |
| Tetrahydrofuran | // | Gel-like |
| 1-propanol/water (98.5:1.5) | // | Gel like |
| 2-propanol/water (96:4) | Form A | Gel to solid |
| Methanol/water (78:22) | Form A | Needle-like particles |
| Methanol/water (57:43 | Form A | |
| Methanol/water (33:67) | // | |

“//”: not enough material

Preparation of Form A from hot saturated solutions at 60° C.: About 100-300 mg of Compound I (or an appropriate amount to ensure saturation) was dissolved in the minimal amount of solvent at about 60° C. The solutions were slowly cooled to ambient temperature under agitation.

If no suspension was obtained after cooling to room temperature or the suspension was too light to collect enough material for analysis, the sample was stored at 5° C. for at least 5 days or for at least 72 hours at −20° C.

The solution was filtered and dried for 10 minutes in air. Precipitation of Compound I under these conditions in a variety of solvents yielded Form A.

TABLE 2
Crystallization of Compound I from hot saturated solutions at 60° C.

| Solvent | XRPD | Comments |
|---|---|---|
| Acetone | Form A | |
| Actonitrile | Form A | |
| Dichloromethane | Form A | |
| Ethanol | Form A | |
| Ethyl acetate | Form A | |
| Isopropyl acetate | Form A | |
| 2-Methyl-2-butanol | // | Clear solution at 4 ° C. for 2 weeks |
| MTBE | Form A | |
| Methanol/water (50:50) | Form A | Needle-like particles |
| Acetone/heptane (50:50) | Form A | |
| Methanol/water (33:67) | Form A | Needle-like particles |
| Methanol/water (42:58) | Form A | Needle-like particles |
| Water | // | No precipitation |

Preparation of Form A by reverse antisolvent addition: A near saturated solution of compound I was added under vigorous agitation into an excess of antisolvent. If there was no immediate precipitation, the mixture was kept stirring at room temperature for a maximum of 24 hours. The precipitate was examined for the polymorphic form.

TABLE 3
Crystallization of Compound I by adding an antisolvent to a solution of
compound I

| Antisolvent | Solvent | Ratio | XRPD | Comments |
|---|---|---|---|---|
| Heptane | Acetone | 1:10 | Form A | Gel to solid |
| Toluene | Acetone | 1:25 | // | Clear solution |
| Water | Acetone | 1:10 | Form A | Gel to solid |
| Heptane | Dioxane | 1:10 | Form A | Not clear to solid |
| Toluene | Dioxane | 1:10 | Form A | Not clear to solid |
| Water | Dioxane | 1:10 | Form A | Not clear to solid |
| Heptane | Ethanol | 1:10 | Form A | Gel to solid |
| Toluene | Ethanol | 1:10 | // | Gel to solid |
| Water | Ethanol | 1:10 | Form A | Gel to solid |
| Heptane | Ethyl acetate | 1:10 | Form A | Gel to solid |
| Toluene | Ethyl acetate | 1:10 | // | Gel to solid |
| Toluene | Methanol | 1:10 | // | Clear solution |
| Water | Methanol | 1:10 | Form A | Turbid to solid |
| Heptane | 2-Methyl-2-butanol | 1:10 | Form A | Gel to solid |
| Toluene | 2-Methyl-2-butanol | 1:10 | // | Gel to gel |
| Water | 2-Methyl-2-butanol | 1:10 | // | Gel to gel |
| Heptane | Tetrahydrofuran | 1:10 | Form A | Turbid to solid |
| Toluene | Tetrahydrofuran | 1:10 | Form A | Turbid to solid |
| Water | Tetrahydrofuran | 1:10 | // | Turbid to solid |

“//”: No precipitation

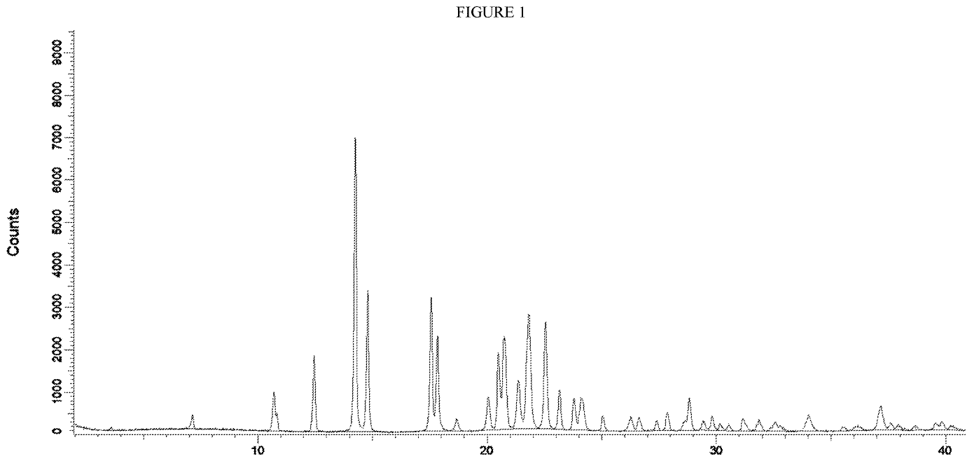

The X-ray powder diffraction pattern of crystalline Form A is shown in FIG. 1 and the peak listing is as shown in Table 4.

TABLE 4
Powder X-Ray Diffraction Peaks compound I Crystal form A

| ° deg 2 0 | d-space | Relative intensity (%) |
|---|---|---|
| 7.1° | 12.39Å | 4% |
| 12.5° | 7.10Å | 26% |
| 14.3º | 6.21Å | 100% |
| 14.8° | 5.98Å | 48% |
| 18.7° | 4.74Å | 4% |
| 20.1° | 4.42Å | 11% |
| 21.8° | 4.07Å | 39% |
| 22.6° | 3.94Å | 36% |
| 23.2° | 3.84Å | 13% |
| 25.1° | 3.55Å | 5% |
| 27.9° | 3.20Å | 6% |

Solubility in solvents: Compound I is soluble with a solubility of greater than 25 mg/ml in many solvents, including acetone, 1,4-dioxane, ethanol, ethyl acetate, isopropyl acetate, methanol, 2-methyl-2-butanol, tetrahydrofuran, and solvent mixtures of 1-propanol/water (98.5:1.5), 2-propanol/water (96:4) and methanol/water (78:22). Compound I is poorly soluble (between 1 and 25 mg/ml) in acetonitrile, dicholormethane, methyl-tert-butyl ether, nitromethane. In toluene and heptane, compound I is sparingly soluble (<1 mg/ml).

Compound I was not observed to form a hydrate when equilibrated at 4° C. in an aqueous fluid, such as water, methanol/water (33:67, 42:58) after 2 weeks and 4 weeks.

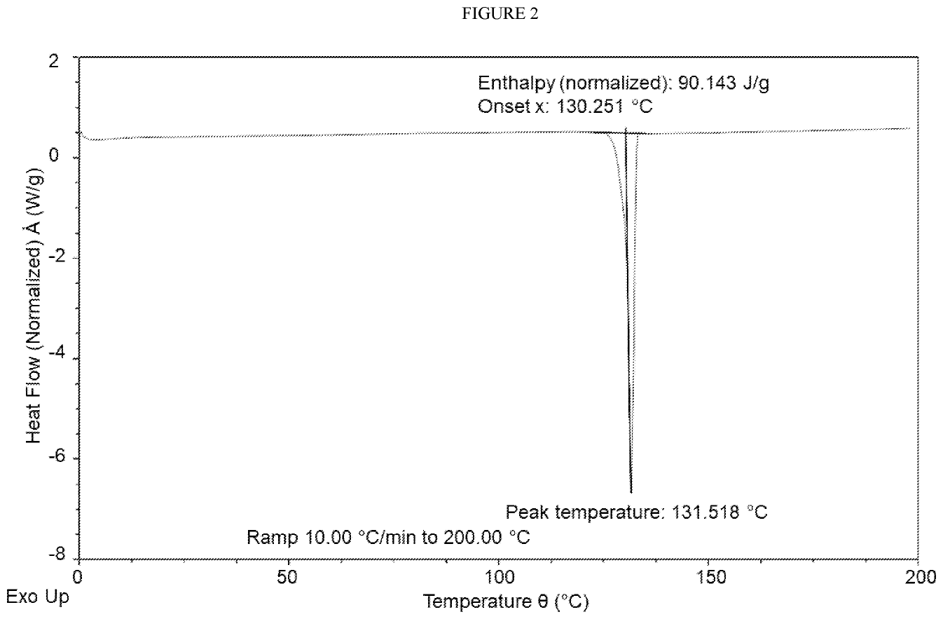

Thermal investigation: Compound I Form A showed a melting point of 130.3° C. with an accompanying enthalpy of fusion of 90 J/g indicating a highly crystalline material (FIG. 2). Melting points varied by only 0.3% and enthalpies by about 3% when varying the heating rate. On cooling and reheating the melt, a glass transition for the amorphous material was found at 40° C. with a change in isobaric heat capacity of 0.54 J/g/K.

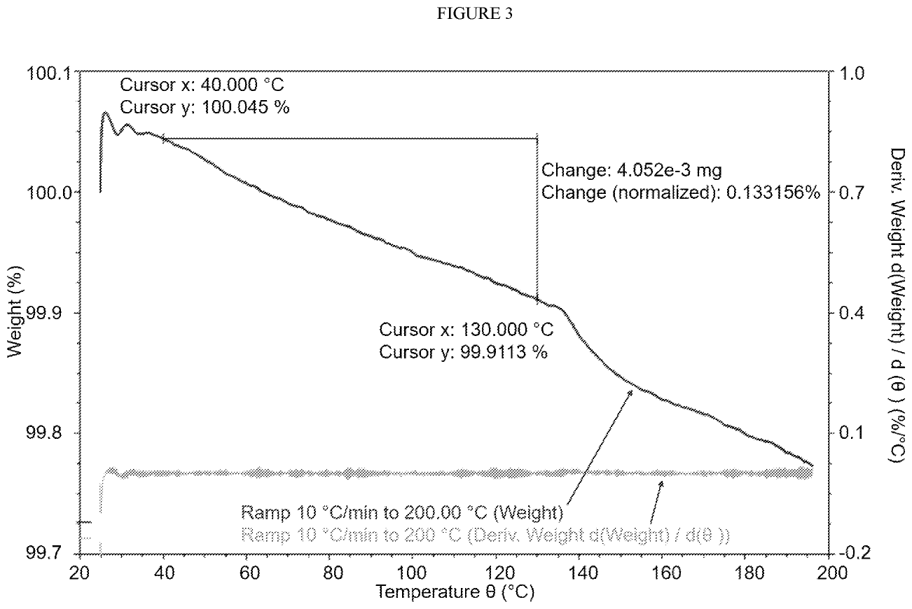

Using TGA, a loss on drying for Form A was determined to be 0.13% at 130° C. (FIG. 3). Exposure of Form A to 92% RH for 24 h did not result in change.

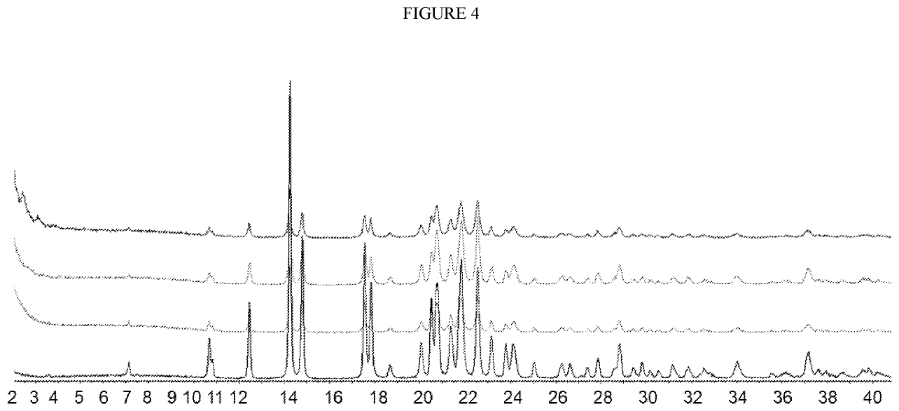

Compound I Form A was slightly hygroscopic by DVS, absorbing approximately 0.4% water vapor at 95% RH with no change in solid state. Further, no form change was observed for Form A after compression, but only minor peak broadening was observed. In addition, Form A did not show form change under dry grinding and wet granulation with water and ethanol (FIG. 4).

### Example 3. Biological Activity of Compound 1 (Example 1)

### Determination of TRPV1 Inhibition

Chinese Hamster Ovary (CHO) cells transfected to express human TrpV1 receptor (which are herein referred to as CHO-huTrpV1 cells), were grown in F-12 Ham's Nutrient Mixture media (HyClone SH30026.01) supplemented with 10% Fetal Bovine Serum (Invitrogen #26140-079), 1% Antibiotic/antimycotic (Invitrogen #15240-062) and 500 μg/mL geneticin (ThermoFisher scientific #1031035). Cells were grown in T-75 flasks at 37° C. incubater with 5% CO₂. The cells were passaged twice a week at a ratio of 1:10 to 1:20 to maintain steady growth. For experimentation, cells were harvarsted at approximately 80% confluency and plated onto 384 well black cell culture plates (cat #781091, Greinier Bio-One Inc.) at 15,000 cells per well in 20 μl media and grown overnight.

### FLIPR Calcium Assay to Detect Calcium Influx in CHO-Hu TrpV1 Cells

The loading dye was prepared following instruction of Calcium 6 assay kit (Molecular Probes, #R8190): 10 ml buffer from bottle B was added to 1 vial of bottle A (adapted to room temperature from −20° C.) and mixed well, then 2.5 mM of fresh prepared probenecid was added and mixed well. 20 μl/well of loading dye was added on top of the cells, and incubated at 37° C. for 1 hour 30 min.

Assay buffer preparation: 1×HBSS, 2 mM HEPES, 0.1% BSA plus 2.5 mM freshly prepared probenecid (Invitrogen, #P36400). 25 μl/well assay buffer in 384 well clear plate (cat #782281, Greiner Bio-one) was dispensed with buffer distributor (Multidrop ComB1 from Thermo Scientific). The compounds were in 384 Echo plate (cat #LPL0200, Labcyte) and the starting concentration of compound was 10 mM, then 1 to 3 serial dilution in 100% DMSO, 8 ul/well). 125 nl compounds was transferred to the 384 well plate containing 25 μl/well buffer with Echo® 555 Liquid Handler (Labcyte), such that the compound concentration was 5 fold of final concentration. The plate was shook slowly at 40 rpm/min for 10 min to mix. 10 μl of 5 fold compound in the buffer was transferred to the cell plate (containing 20 μl cells and 20 μl dye) using Vertical Pipetting Station 384ST (Agilent Technologies). Six fold of final concentration of NADA(N-arachidonyl dopamine, cat #A8848, Sigma) in the assay buffer was prepared and 30 μl/well was distributed in 384 well clear plate.

After compounds were added to the cell plate with loading dye, within 10-15 minutes, cell plate and plate containing NADA was placed into the FLIPR (Fluorescent Imaging Plate Reader) instrument (Tetra System, Molecular device). The TRPV1 receptor was stimulated by application of 10 μl per well of NADA. For testing the effect of compounds for possible antagonism, 2.5 μM NADA was used at the EC80 concentration.

For determination of antagonist IC₅₀ values (concentration of antagonist that inhibits response to NADA by 50%), at least 10 antagonist concentration were measured in triplicate. The response in the presence of the antagonist was calculated as a percentage of the control response to NADA and was plotted against the concentration of the antagonist. The IC₅₀ was estimated by non-linear regression analysis to sigmoidal-logistic curves by HELIOS (PROD 2) system. These values were averaged (means and standard error of the mean) for at least three independent experiments.

TABLE 5
Antagonist effect of compound (I) against human TRPV1

| | IC50 |
|---|---|
| Example Number | (μM) |
| Example 1 (racemic) | 0.0104 |
| Example 1: (S)-1-(6-fluoro-5-((((1r, 3S)-3-(4-fluoro-3- | 0.00062 |
| (trifluoromethyl)phenoxy)cyclobutyl)amino)methyl)isoquinolin- | |
| 8-yl)ethane-1,2-diol | |
| Example 1: (R)-1-(6-fluoro-5-((((1r, 3R)-3-(4-fluoro-3- | 0.0063 |
| (trifluoromethyl)phenoxy)cyclobutyl)amino)methyl)isoquinolin- | |
| 8-yl)ethane-1,2-diol | |

All publications and patent documents cited herein are incorporated herein by reference as if each such publication or document was specifically and individually indicated to be incorporated herein by reference. The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the invention that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein. The claims should not be read as limited to the described order or elements unless stated to that effect. It should be understood that various changes in form and detail may be made without departing from the scope of the appended claims.

What is claimed is:

1. A crystalline form of 1-(6-fluoro-5-((((1r,3r)-3-(4-fluoro-3-(trifluoromethyl)phenoxy)cyclobutyl)amino)methyl)isoquinolin-8-yl)ethane-1,2-diol (compound I) having structure

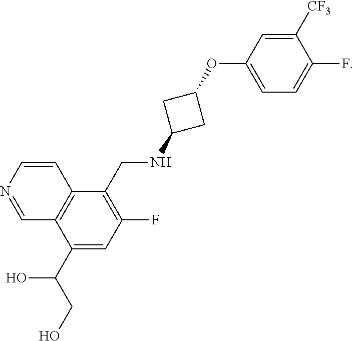 [SMILES: OCC(O)c1cc(F)c(CN[C@H]2C[C@H](Oc3ccc(F)c(C(F)(F)F)c3)C2)c2ccncc12]

2. The crystalline form of compound I according to claim 1, having the structure

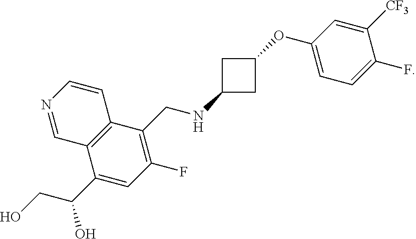 [SMILES: OC[C@@H](O)c1cc(F)c(CN[C@H]2C[C@H](Oc3ccc(F)c(C(F)(F)F)c3)C2)c2ccncc12]

3. The crystalline form of compound I according to claim 2, having a crystalline Form A.

4. The crystalline form of a compound of formula I according to any of claims 1-3, characterized by an X ray diffraction pattern having three or more peaks at 2θ values selected from 14.3, 14.8, and 21.8±0.2 °2θ.

5. The crystalline form of compound I according to any of claims 1-4, characterized by an X ray diffraction pattern having three or more peaks at 2θ values selected from 12.5, 14.3, 14.8, 21.8, and 22.6±0.2 °2θ.

6. The crystalline form of compound I according to any of claims 1-5, characterized by an X ray diffraction pattern having 3 or more, 4 or more, 5 or more, 6 or more, or 7 or more peaks at 2θ values selected from 12.5, 14.3, 14.8, 20.1, 21.8, 22.6, and 23.2±0.2 °2θ.

7. The crystalline form of compound I according to any of claims 1-6, characterized by an X ray diffraction pattern as shown in FIG. 1.

8. The crystalline form of compound I according to any of claims 1-7, characterized by an differential scanning calorimetry pattern as shown in FIG. 2.

9. The crystalline form of Compound I according to any of claims 1-8, characterized by a DSC thermogram exhibiting an endotherm at about 131.5° C.

10. The crystalline form of Compound I according to any of claims 1-9, characterized by a water loss as measured by thermogravimetric analysis of about 0.13 wt. %.

11. The crystalline form of Compound I according to claim 10, characterized by a water loss as measured by thermogravimetric analysis of about 0.13 wt. % at 130° C.

12. A method of preparing a crystalline Form A of compound I according to any of claims 1-11, comprising cooling a hot saturated solution of compound I in a solvent, to crystallize compound I as crystal form A.

13. The method of claim 12, wherein the solution is cooled to a temperature of between about 0° C. to about 25° C.

14. The method of claim 12 or 13, wherein the solvent is selected from the group consisting of acetone, acetonitrile, dichloromethane, ethanol, ethyl acetate, isopropyl acetate, methyl tert-butyl ether, acetone/heptane, and methanol/water.

15. A method of preparing a crystalline Form A of compound I according to any of claims 1-11, comprising crystallizing form A from a solution of compound I in a solvent.

16. The method of claim 15, wherein the solution is saturated.

17. The method of any of claims 15-16, wherein the form A is crystallized at a temperature of about 25° C.

18. The method of any of claims 15-17, wherein the solvent is selected from the group consisting of acetone, acetonitrile, dichloromethane, ethyl acetate, isopropyl acetate, 2-methyl-2-butanol, and methyl tert-butyl ether.

19. The method of any of claims 15-17, wherein the solvent is selected from the group consisting of mixtures of water/methanol, water/1-propanol, and water/2-propanol.

20. A method of preparing a crystalline Form A of compound I according to any of claims 1-11, comprising adding an antisolvent to a solution of compound I in a solvent.

21. The method of claim 20, wherein the solvent is selected from the group consisting of acetone, dioxane, ethanol, ethyl acetate, methanol, 2-methyl-2-butanol, and tetrahydrofuran.

22. The method of claim 20 or claim 21, wherein the antisolvent is selected from the group consisting of water, heptane, and toluene.

23. A composition, comprising a crystalline form of compound I according to any of claims 1-11.

24. A composition, comprising a crystalline Form A of compound I in substantially pure form.

25. Use of a crystalline form of compound I according to any of claims 1-11, in the manufacture of a medicament for the treatment of ocular surface pain.

26. Use of a crystalline form of compound I according to any of claims 1-11, in the manufacture of a medicament for the treatment of an ocular surface disorder.

27. Use of a crystalline form of Compound I according to any of claims 1-11, in the preparation of a pharmaceutical formulation.

28. A method of preparing a pharmaceutical formulation comprising a crystalline form of Compound I according to any of claims 1-11, the method comprising dissolving the crystalline form of Compound I in an ophthalmically acceptable carrier formulated for ocular use, e.g., for topical application to the ocular surface.
